(19) United States
(12) Patent Application Publication
(10) Pub. No.: US 2009/0204009 A1
(43) Pub. Date: Aug. 13, 2009

(54) MEDICAL DEVICE SYSTEM AND RELATED METHODS FOR DIAGNOSING ABNORMAL MEDICAL CONDITIONS BASED ON IN-VIVO OPTICAL PROPERTIES OF TISSUE

(75) Inventors: Tamara M. Powers, Los Alamos, NM (US); Judith R. Mourant, Los Alamos, NM (US); Robert E. Hermes, Los Alamos, NM (US)

Correspondence Address: JOHN P. O'BANION;O'BANION & RITCHEY LLP, 400 CAPITOL MALL SUITE 1550, SACRAMENTO, CA, 95814, US

(73) Assignee: LOS ALAMOS NATIONAL SECURITY, Los Alamos, NM (US)

(21) Appl. No.: 12/030,835

(22) Filed: Feb. 13, 2008

Related U.S. Application Data
(60) Provisional application No. 61/026,921, filed on Feb. 7, 2008.

(51) Int. Cl. A61B 6/00

(52) U.S. Cl. 600/476

(57) ABSTRACT

A diagnostic system detects abnormal physical properties of tissue in a patient based upon optical properties of the tissue. A probe includes light delivery and capture fibers, and polarizers. Optical properties detected relate to polarized and unpolarized light into, and scattering from, the tissue. The optical properties detected are processed and analyzed to produce results indicative of the physical property(s) evaluated. System operation is controlled against pressure monitored via a pressure sensor coupled to the probe-tissue interface. The analysis corrects for patient physical characteristics as user inputs, such as menopausal or menstrual condition of women patients. Physical properties diagnosed include cervical dysplasia conditions in women patients, such as HSIL, cervical cancer, LSIL, or cervicitis. Analysis and diagnosis is based upon at least one of: ratios between scattered light signals captured from the tissue, slope of intensity over wavelength for scattered light signals captured, and hemoglobin-related parameters in the tissue.

### CROSS-REFERENCE TO RELATED APPLICATIONS

This application claims priority to copending U.S. Provisional application No. 61/026,921, filed on Feb. 7, 2008, incorporated herein by reference in its entirety.

### STATEMENT REGARDING FEDERALLY SPONSORED RESEARCH OR DEVELOPMENT

This invention was made with Government support under Grant No. 6R01CA071898-09, awarded by the National Institute of Health (NIH) and National Cancer Institute (NCI) under program code RAEF B&R 4004412000. The Government has certain rights in this invention.

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C. F. R. § 1.14.

### BACKGROUND OF THE INVENTION

1. Field of the Invention

This invention pertains generally to medical device systems and methods for detecting certain properties of tissue in patients. More particularly, it relates to diagnosing tissue abnormalities. Still more particularly, it relates to such systems and methods for diagnosing cancerous or precancerous conditions in tissue based upon certain optical properties of the tissue.

2. Description of Related Art

The American Cancer Society estimates that, in 2006, 9,710 cases of invasive cervical cancer will be diagnosed in the United States and 3,700 women will die from this disease. In the United States and western Europe, mortality from cervical carcinoma has significantly decreased coincident with the wide spread use of the Papanicolaou test (Pap smear) followed by colposcopy and detection of preinvasive and early stage disease. However, there are many limitations to currently acceptable screening and diagnostic strategies.

From a clinical prospective, it is particularly beneficial and valuable to distinguish those pre-invasive lesions likely to progress to invasive carcinoma if left untreated in a cost-efficient manner. The Pap smear test frequently has a low sensitivity; high sensitivity and specificity are not achieved concurrently. Additionally, neither the Pap smear nor colposcopy-directed biopsy provide real-time diagnostic information. The patient must be contacted later to learn the results and set-up any future treatment/examinations. In the inner city clinics, up to 70% of patients with high grade lesions do not complete recommended follow-up examinations. “See and treat” protocols in which a loop electrosurgical excision procedure (or “LEEP” procedure) can be performed at the time of initial colposcopy have been proposed so that patients need not return for treatment. However inaccuracies of Pap smear results and colposcopic impression often lead to over treatment. There is a pressing need for improved diagnostics in order for “see and treat” protocols to reach their potential.

There are many methods under investigation to reduce screening and surveillance costs and improve detection of high grade squamous intraepithelial lesions (HSIL) which are a cervical cancer precursor. These include testing for human papilloma viruses (HPV) that are known to be associated with cervical cancer as well as several non-intrusive optical and optoelectronic methods. HPV tests have been shown to have high clinical sensitivity to HSIL; however, less than 10% of women with HPV have or will develop cervical intraepithelial neoplasia (CIN) III over a prospective 3 to 4 year time frame.

Various optical techniques have been developed in order to evaluate certain properties of tissues in patients. Several such approaches have been intended to assist in diagnosing abnormal properties in tissue. The basis for some optical techniques has been to detect biochemical and morphological features that are concurrent with precancerous conditions. Examples of optical spectroscopy methods are elastic light scattering, fluorescence, optical coherence tomography, and Raman spectroscopy. Fluorescence and Raman spectroscopy are primarily sensitive to biochemical changes, while light scattering and optical coherence tomography are primarily sensitive to morphological changes. However, in any case, various shortcomings of prior optical approaches are readily apparent.

There is a need for improved diagnostic systems and methods for accurately and repeatably detecting abnormal tissue conditions in a manner that is efficient with respect to patient management and associated costs.

There is a need for improved diagnostic systems and methods that readily diagnose abnormal conditions in tissue sufficiently to enable “see-and-treat” protocols without substantial risk of over or under treatment across a wide range of patients.

There is in particular such a need for diagnosing cancerous and pre-cancerous conditions, and other forms of tissue dysplasia, including in particular as related to abnormal cervical conditions in women such as for example HSIL, cervical cancer, low grade squamous intraepithelial lesions (LSIL), and cervicitis.

There is also a particular need for improved systems and methods for measuring optical properties of tissue in a manner that may be efficiently used to accurately and repeatably diagnose abnormal conditions in the tissue over a wide range of patients and related physical characteristics.

### BRIEF SUMMARY OF THE INVENTION

One aspect of the present disclosure provides an improved diagnostic system and method for accurately and repeatably detecting abnormal tissue conditions in a manner that is efficient with respect to patient management and associated costs.

Another aspect of the present disclosure provides an improved diagnostic system and method that readily detects abnormal conditions in tissue sufficiently to enable “see-and-treat” protocols without substantial risk of over or under treatment across a wide range of patients.

Another aspect of the present disclosure provides an improved system and method for diagnosing cancerous and pre-cancerous conditions, and other forms of tissue dysplasia, including according to certain particular modes as related to abnormal cervical conditions in women such as for example HSIL, cervical cancer, LSIL, and cervicitis.

Another aspect of the present disclosure provides an improved system and method for measuring optical properties of tissue in a manner that may be efficiently used to accurately and repeatably diagnose abnormal conditions in the tissue over a wide range of patients and related physical characteristics.

Another aspect is a medical diagnostic system that includes a probe with a proximal end portion and a distal end portion with a tissue interface region. At least one light delivery member is coupled to the tissue interface region, at least one light capture member is coupled to the tissue interface region; and a pressure sensor is also coupled to the tissue interface region.

According to one mode, the system also includes a light illumination system coupled to the at least one light delivery member, an optical measurement system coupled to the at least one light capture member and a pressure monitoring system coupled to the pressure sensor. The system is adjustable between an “off” mode and an operating mode when the tissue interface region is in contact with a region of tissue of a patient. In at least the operating mode the light illumination system illuminates the at least one light delivery member to emit at least one incident light signal from the tissue interface region into the region of tissue, the optical measurement system measures at least one property of at least one captured light signal collected from the region of tissue by the at least one light capture member at the tissue interface region, and the pressure monitoring system monitors a pressure at the tissue interface region in contact with the region of tissue.

In one embodiment, a controller is coupled to the pressure monitoring system. The controller controls (a) at least one aspect of the operating mode of the system, and/or (b) an indicator that provides pressure-dependent indicia to a user useful in controlling at least one aspect of the operating mode of the system, based upon the pressure measured by the pressure monitoring system.

In a further embodiment, an algorithm is accessed by the controller that determines a result based upon the measured pressure. The result is used by the controller to control the operating mode of the system or to provide the pressure-dependent indicia to the user in order to manually control the operating mode of the system.

In still a further embodiment, the algorithm comprises a pre-determined pressure criteria associated with a binary “on-off” decision, such that a measured pressure meeting the criteria is associated with one of an “on” or “off” decision, and a measured pressure not meeting the criteria is associated with the other of the “on” or “off” decision. According to an “on” decision by the algorithm, the controller actuates the system into the operating mode or actuates the indicator to provide indicia to a user to adjust the system into the operating mode. According to an “off” decision by the algorithm, the controller either does not actuate the system into the operating mode or controls the indicator to indicate to the user the “off” decision not to adjust the system to the operating mode.

In still another further embodiment, the pressure criteria comprises a first pressure threshold, such that a measured pressure above the first pressure threshold corresponds with an “on” decision by the algorithm, and a measured pressure below the first pressure threshold corresponds with an “off” decision by the algorithm.

In another further embodiment, the pressure criteria comprises a first pressure threshold, such that a measured pressure below the first pressure threshold corresponds with an “on” decision by the algorithm, and a measured pressure above the first pressure threshold corresponds with an “off” decision by the algorithm. In a further variation of this embodiment, the pressure criteria also comprises a second pressure threshold, the second pressure threshold is lower than the first pressure threshold, such that a pressure range criteria is provided between the first and second pressure thresholds, and such that a measured pressure within the pressure range criteria corresponds with an “on” decision by the algorithm, and a measured pressure outside of the pressure range criteria corresponds with an “off” decision by the algorithm. In still a further variation, the second pressure threshold is greater than zero and less than about 1 psi. In another variation, the second pressure threshold is between about 0.5 psi and about 1 psi. In still another variation of the present embodiments, the first pressure threshold is equal to or less than about 3 psi, and may be less than about 2 or 1 psi.

According to another embodiment, a processor is coupled to the controller and also to the optical measurement system. In the operating mode, the processor processes information corresponding with the at least one property measured by the optical measurement system and produces a result that is useful to a user in performing a medical diagnosis on the patient.

According to one variation of this embodiment, the information is related to presence of cancerous or pre-cancerous tissue in the region of tissue, and the result is useful to a user in diagnosing the presence of cancerous or pre-cancerous tissue in the region of tissue. In a further variation, the probe comprises a cervical probe, the information is related to presence of cervical cancer or cervical pre-cancerous tissue in the region of tissue, and the result is useful to a user in diagnosing the presence of cervical cancer or cervical pre-cancerous tissue in the region of tissue. In another embodiment, the information is related to presence of HSIL in the region of tissue, and the result is useful to a user in diagnosing the presence of HSIL in the region of tissue.

According to another embodiment, an indicator is coupled to and controlled by the controller so as to provide an indication useful to a user in controlling the operating mode of the system based upon the measured pressure. The indicator may comprise for example at least one of a visual indicator and a sound indicator.

In another embodiment, the system includes a first light delivery member with a distal end comprising a first light emitter at the tissue interface region and optically coupled to the light illumination system, a second light delivery member with a distal end comprising a second light emitter at the tissue interface region and optically coupled to the light illumination system, a first light capture member with a distal end comprising a first light collector at the tissue interface region and optically coupled to the optical measurement system, and a second light capture member with a distal end comprising a second light collector at the tissue interface region and optically coupled to the optical measurement system. The controller in the operating mode controls the system such that the light illumination system illuminates the first light delivery member and second light delivery member independently during first and second unique time sequences, respectively, and such that the optical measurement system is controlled to acquire data from the first and second light capture members independently and uniquely during the unique time sequences, also respectively, corresponding with illumination of the respective light delivery members.

In one further variation considered particularly beneficial, the system further includes a third light capture member with a distal end comprising a third light collector at the tissue interface region and optically coupled to the optical measurement system, a fourth light capture member with a distal end comprising a fourth light collector at the tissue interface region and optically coupled to the optical measurement system, a first polarizer located over the first light delivery member and the first and fourth light capture members at the tissue interface region; and a second polarizer located over the third light capture member at the tissue interface region. The second light delivery member and second light capture member are each uncovered and unpolarized at the tissue interface region. The second polarizer has a different polarization than the first polarizer. The controller in the operating mode controls the optical measurement system and processor to recognize unique light signal data captured at the first, third and fourth light capture members during illumination of a respective light delivery member.

Still further to this variation, a processor may be coupled to the optical measurement system and controlled by the controller to process optical information in a manner that produces a diagnostically useful result based upon a parameter associated with at least one of: a ratio of light captured at the first and third light capture members, a ratio of light captured at the first and fourth light capture members, slope of at least one captured light signal or ratio over wavelength, average distance light traveled through the tissue, total hemoglobin, and total oxygenated hemoglobin, at least one further ratio of one or more of the foregoing against reference calibration light measurements taken with the probe, and combinations thereof. According to a further beneficial feature that may also be included, the processor is configured to process the information and produce the result in a variable manner based upon at least one patient history or patient health parameter input.

Another aspect of the present disclosure is a method for diagnosing a property of a region of tissue in a patient that includes placing a tissue interface region of a probe in contact with the region of tissue, delivering at least a first light illumination signal from the tissue interface region into the region of tissue in contact therewith, collecting at least a first captured light signal from the region of tissue at the tissue interface region in response to the first light illumination signal delivered into the region of tissue, processing at least one measured parameter of at least the first captured light signal and producing a result that is useful in diagnosing the property of the region of tissue, monitoring a pressure at the tissue interface region in contact with the region of tissue, comparing the monitored pressure against a pressure threshold criteria, and producing the result only based upon the monitored pressure meeting the pressure threshold criteria.

Another aspect of the present disclosure is a medical diagnostic system that includes an optical measurement system configured to measure at least one light scattering property or light absorption property of tissue, a data analysis algorithm, and a processor coupled to the data analysis algorithm. The data analysis algorithm run by the processor is configured to analyze data related to the measured optical property or properties in a manner that provides output information that is useful in diagnosing a property of the tissue. In addition, the data analysis algorithm adjusts the analysis and output information based upon physical characteristics of the patient input by the user.

Another aspect of the present disclosure is a method for diagnosing a physical property of a region of tissue in a patient, comprising: measuring an optical property of a region of tissue in a patient; and monitoring a pressure associated with the region of tissue being measured.

Another aspect of the present disclosure is a medical diagnostic system, comprising: an optical measurement system configured to measure an optical property of a region of tissue in a patient; a software algorithm stored in a computer readable medium; a computer-based user interface; and a processor that is coupled to the optical measurement system in a manner that receives the measured optical property, and to the computer readable medium in a manner configured to run the software, and also to the computer-based user interface. The software algorithm run by the processor is configured to analyze data related to the measured optical property in a manner that provides output information that is useful in diagnosing a property of the tissue. In addition, the software algorithm adjusts the analysis and output information based upon at least one physical characteristic of the patient input by a user via the computer-based user interface.

Another aspect of the present disclosure is a medical diagnostic system, comprising: an optical measurement system configured to receive and measure at least one light scattering property of tissue in response to at least one of a first polarized light illumination signal with a first polarization and a second unpolarized light illumination signal into the tissue from at least one light source; a software algorithm stored on a computer readable medium; and a processor coupled to the optical measurement system and also to the computer readable medium in a manner adapted to run the software. The software run by the processor calculates at least one value associated with at least one of the following parameters over a range of wavelength of illuminating light into the region of tissue: (a) a ratio between first and second scattered light signals captured at first and second separate locations, respectively, through at least a first polarizer that passes light aligned with the first polarization and filters light with a second polarization that is perpendicular to the first polarization; (b) a ratio between the first scattered light signal captured at the first location through the first polarizer and a third scattered light signal captured at a third location that is different from the first and second locations and through a second polarizer that passes light with the second polarization and filters light with the first polarization; (c) a slope of signal intensity over a range of wavelength for a fourth scattered light signal from the tissue in response to unpolarized illumination of the tissue and captured at a fourth location that is different from the first, second, and third locations; (d) total oxygenated hemoglobin in the tissue; (e) total deoxygenated hemoglobin in the tissue; and (f) average distance light traveled through the tissue.

Another aspect of the present disclosure is a method for diagnosing a physical property of a region of tissue in a patient, comprising: measuring an optical property of the region of tissue in the patient; analyzing data related to the measured optical property; and providing output information based upon the data analysis that is useful in diagnosing the physical property of the tissue. The analysis and output information is based at least in part upon at least one input parameter associated with a physical characteristic of the patient.

Another aspect of the present disclosure is a method for diagnosing a physical property of a region of tissue in a patient, comprising: illuminating the region of tissue with at least one of a first polarized light illumination signal with a first polarization and a second unpolarized light illumination signal from at least one light source; measuring at least one light scattering property of tissue in response to at least one of the first polarized light illumination signal and the second unpolarized light illumination signal into the tissue; and calculating at least one value associated with at least one of the following parameters over a range of wavelength of illuminating light into the region of tissue: (a) a ratio between first and second scattered light signals captured at first and second separate locations, respectively, through at least a first polarizer that passes light aligned with the first polarization and filters light with a second polarization that is perpendicular to the first polarization; (b) a ratio between the first scattered light signal captured at the first location through the first polarizer and a third scattered light signal captured at a third location that is different from the first and second locations and through a second polarizer that passes light with the second polarization and filters light with the first polarization; (c) a slope of signal intensity over a range of wavelength for a fourth scattered light signal from the tissue in response to unpolarized illumination of the tissue and captured at a fourth location that is different from the first, second, and third locations; (d) total oxygenated hemoglobin in the tissue; (e) total deoxygenated hemoglobin in the tissue; and (f) average distance the light traveled through the tissue.

Another aspect of the present disclosure is a method for diagnosing a physical property of a region of tissue in a patient, comprising: measuring an optical property of the region of tissue in the patient; analyzing data related to the measured optical property; and providing output information based upon the data analysis that is useful in diagnosing the physical property of the tissue. The analysis and output information provides indicia of one of the following three categories: (a) presence of the physical property; (b) not known; and (c) absence of the physical property.

Another embodiment of the various aspects, modes, embodiments, and variations or features noted above further includes correcting the diagnostic parameters processed from the optical measurements for known physiological effects. According to one specific embodiment, measurements of patients with the same diagnosis that depend on whether the patient is menopausal or not are correlated with such input. Another further embodiment comprises combining corrected parameters into a diagnostic algorithm. In one regard, a voting method is used. In one particular embodiment, a clustering method based on Mahalanobis distance is employed. In further embodiments, logistic regression or SIMCA is employed. The outcomes of the diagnostic algorithm can be for example in one particular beneficial embodiment: (a) HGSIL or cancer; (b) not known; or (c) diagnoses other than HSIL and cancer (i.e. LSIL, cervicitis etc.).

According to another present embodiment of this disclosure, an output of a diagnostic algorithm is provided in terms of disease likelihood (i.e. not a yes no answer, but a probability answer).

Another aspect of the present disclosure is a medical diagnostic system, comprising: a probe with a proximal end portion and a distal end portion providing a tissue interface; at least one light delivery member coupled to the distal end portion at the tissue interface; at least one light capture member coupled to the distal end portion at the tissue interface; and a pressure sensor coupled to the tip at the tissue interface.

Another aspect is a medical diagnostic system, comprising an optical measurement system configured to measure an optical property of a region of tissue in a patient, and a pressure monitoring system configured to monitor a pressure associated with the region of tissue measured by the optical measuring system.

Another aspect of the present disclosure is a method for diagnosing a property of a region of tissue in a patient, comprising: delivering at least a first light illumination signal into the region of tissue; capturing at least a first scattered light signal from the region of tissue in response to the first light illumination signal delivered into the region of tissue; and sensing a pressure associated with the region of tissue while capturing the first scattered light signal.

Another aspect of the present disclosure is a method for diagnosing a physical property of a region of tissue in a patient, comprising: measuring an optical property of a region of tissue in a patient; and monitoring a pressure associated with the region of tissue being measured.

Another aspect of the present disclosure is a medical diagnostic system, comprising: an optical measurement system configured to measure at least one light scattering property or light absorption property; a data analysis algorithm; a processor coupled to the optical measurement system and able to implement the data analysis algorithm. The data analysis algorithm run by the processor is configured to analyze data related to the measured optical property or properties in a manner that provides output information that is useful in diagnosing a property of the tissue. The data analysis algorithm also adjusts the analysis and output information based upon physical characteristics of the patient input by a user.

Another aspect of the present disclosure is a medical diagnostic system, comprising: an optical measurement system configured to receive and measure at least one light scattering property or light absorption property of tissue in response to at least one of a first polarized light illumination signal with a first polarization and a second unpolarized light illumination signal into the tissue from at least one light source; a data analysis algorithm; and a processor coupled to the optical measurement system and able to implement the data analysis algorithm. The processor running the data analysis algorithm calculates at least one of a parameter that closely approximates the average distance light traveled in tissue multiplied by the total oxygenated hemoglobin in tissue and a parameter that closely approximates the average distance light traveled in tissue multiplied by the total deoxygenated hemoglobin in tissue.

Another aspect of the present disclosure is a method for diagnosing one or more properties of a region of tissue in a patient comprising: measuring one or more light scattering or light absorption properties in the region of tissue in the patient; analyzing data related to the measured optical properties; and providing output information based upon the data analysis that is useful in diagnosing the tissue property or properties.

Another aspect of the present disclosure is a method for diagnosing a physical property of a region of tissue in a patient, comprising: illuminating the region of tissue with at least one of a first polarized light illumination signal with a first polarization and a second unpolarized light illumination signal from at least one light source; measuring at least one light scattering property of tissue in response to at least one of the first polarized light illumination signal and the second unpolarized light illumination signal into the tissue; and calculating a value associated with at least one of the following parameters over a range of wavelength of illuminating light into the region of tissue: (a) a ratio between first and second scattered light signals captured at first and second separate locations, respectively, through at least a first polarizer that passes light aligned with the first polarization and filters light with a second polarization that is perpendicular to the first polarization; (b) a ratio between the first scattered light signal captured at the first location through the first polarizer and a third scattered light signal captured at a third location that is different from the first and second locations and through a second polarizer that passes light with the second polarization and filters light with the first polarization; (c) a slope of signal intensity over a range of wavelength for a fourth scattered light signal from the tissue in response to un-polarized illumination of the tissue and captured at a fourth location that is different from the first, second, and third locations; (d) total oxygenated hemoglobin in the tissue; (e) total deoxygenated hemoglobin in the tissue; and (f) average distance light traveled through the tissue.

Another aspect of the present disclosure is a method for diagnosing one or more properties of a region of tissue in a patient comprising: measuring one or more optical properties of the region of tissue in the patient; analyzing data related to the measured optical properties; and providing output information based upon the data analysis that is useful in diagnosing the physical property of the tissue. The analysis and output information provides indicia of one of multiple categories of tissue types.

Another aspect of the present disclosure is a probe having a tip with a tissue interface region that is placed in contact with a region of tissue to be diagnosed, and having a pressure sensor coupled to the tissue interface region and also a diagnostic system with a diagnostic sensor coupled to the tissue interface region that is configured to acquire a diagnostically useful signal and perform a diagnostically useful measurement of a parameter associated with the region of tissue. In one mode, the pressure sensor monitors pressure while the diagnostically useful signal and measurement are taken. In another mode, the pressure sensor monitors pressure at the tissue interface that is used to control at least one aspect of the operating mode of the diagnostic system. In another mode, the diagnostic system is an optical measurement system and the diagnostic sensor is a light capture member or fiber. In a further embodiment, a light illumination system and light delivery member are coupled to the probe to emit light from the tissue interface region. In another embodiment, at least a portion of the light is captured through a polarizer. In another embodiment at least a portion of the light emitted into the tissue region is polarized. In still another embodiment, at least one polarizer is provided at the tissue interface region, whereas in still a further embodiment two polarizers are provided at the tissue interface region and with different (e.g. opposite or perpendicular) polarization. In another mode, the diagnostic system is an electrical system and the diagnostic sensor acquires an electrical property of the tissue.

According to further modes of various aspects noted hereunder, optical information acquired by a system for data analysis and diagnosis purposes may include acquired properties during “light on” and “light off” conditions. In further embodiments the data acquired for analysis and diagnosis may also include reference calibration acquisition steps and related materials included in an overall system.

Each of the foregoing aspects, and related modes, embodiments, variations, and features thereof, is considered of independent value and benefit as an invention presented hereunder, as are the various combinations therebetween as presented throughout this disclosure and otherwise readily apparent to one of ordinary skill having reviewed these disclosed contents. Further aspects of the present disclosure and inventions contained hereunder will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1A shows an end-view of a schematic layout of a probe tip according to one embodiment of the present disclosure.

FIG. 1B shows a side-view of the probe tip shown in FIG. 1A, with certain features within the probe tip shown in shadow.

FIG. 1C shows another side-view from a second orientation perpendicular to the first orientation of the probe tip shown in FIGS. 1A-B, with certain features within the probe tip shown in shadow.

FIG. 2 shows a schematic side-view layout of certain features associated with the probe tip shown in FIGS. 1A-C.

FIG. 3 shows a schematic side-view of a probe incorporating a similar probe tip as shown in FIGS. 1A-2, and with other features proximal to the probe tip shown schematically including certain features shown in shadow within the probe.

FIG. 4 shows a schematic layout of a diagnostic system incorporating a probe similar to that shown in various views in FIGS. 1A-3, and including other cooperating components in the system.

FIG. 5 shows a graph of unpolarized light signal intensity reflected from tissue over a range of wavelength according to one mode of using a diagnostic system similar to that shown in FIG. 4.

FIG. 6 shows another graph of polarized light signal intensity reflected from tissue over a range of wavelength according to further modes of using a diagnostic system similar to that shown in FIG. 4, including as measured from three different light capture fibers during on and off light delivery conditions in tissue.

FIG. 7 shows another graph of unpolarized light signal intensity reflected from tissue over a range of wavelength, according to still another mode of using a diagnostic system similar to that shown in FIG. 4, and shows exemplary results indicating a presence of HSIL.

FIG. 8 shows another graph of the ratio I1/I3 of light signal intensity reflected from tissue as measured at each of two light capture fibers 1 and 3 over a range of wavelength, according to still another mode of using a diagnostic system similar to that shown in FIG. 4, and also shows exemplary results indicating a presence of HSIL.

FIG. 9 shows another graph of the ratio I1/I4 of light signal intensity reflected from tissue as measured at each of light capture fibers 1 and 4 over a range of wavelength, according to still another mode of using a diagnostic system similar to that shown in FIG. 4, and also shows exemplary results indicating a presence of HSIL.

FIGS. 10A-C shows various aspects of another diagnostic system according to the present disclosure and used according to an experimental study performed under Example 1, wherein FIG. 10A shows a schematic view of three cooperating components within the diagnostic system, FIG. 10B shows a schematic end-view of a light probe used in the system, and FIG. 10C shows a schematic side-view of certain features of the probe's tip at a tissue interface.

FIGS. 11A-C show three graphs of certain monitored light signal intensities reflected from tissue over a range of wavelength during certain modes of using a probe in a diagnostic system as represented schematically in FIGS. 10A-C and according to the experimental study performed under Example 1, wherein FIG. 11A shows Normalized unpolarized intensity, FIG. 11B shows the ratio I1/I3 of intensities taken at light capture fibers 1 and 3 shown in FIG. 10B, and FIG. 11C shows the ratio I1/I4 of intensities taken at light capture fibers 1 and 4 of the probe shown in FIG. 10B.

FIG. 12 shows a histogram of the ratios of I1/I4 intensities taken at fibers 1 and 4 of the probe shown in FIG. 10(b) for various categories of tissue types according to another aspect of the experimental study performed under Example 1.

FIG. 13 shows a graph of Sensitivity versus 100-Specificity for certain data analysis performed in the experimental study under Example 1.

FIG. 14 shows another graph of Sensitivity versus 100-Specificity for certain other data analysis performed in the experimental study under Example 1.

FIGS. 15A-D show Sensitivity versus 100-Specificity for certain data analysis performed in another experiment conducted under Example 2, with FIG. 15A showing slope, FIG. 15B showing I1/I3 ratio, FIG. 15C showing I1/I4 ratio, and FIG. 15D showing total Hb.

FIGS. 16A-B show two graphs under two panes, FIG. 16A and FIG. 16B, respectively, for Probability of certain tissue types over a range of certain parameters analyzed under the experiment conducted under Example 2, wherein FIG. 16A shows Probability versus ratio I1/I4 of light intensity reflected from tissue taken at two light capture fibers 1 and 4 from a probe, and FIG. 16B shows Probability versus slope.

FIG. 17A shows a graph of certain data analyzed according to another experiment conducted under Example 3 hereunder using a probe and diagnostic system similar to that shown in various aspects in FIGS. 1A-4, and compares slope of reflected light signal intensity along a particular range of wavelengths versus pressure of a monitoring probe against the tissue being monitored.

FIG. 17B shows a graph of certain other data analysis performed in the experiment conducted under Example 3, except showing ratios I1/I3 and I1/I4 for reflected light intensity taken at light capture fibers 1 and 3, and 1 and 4, respectively, versus probe pressure against the tissue being monitored.

### DETAILED DESCRIPTION OF THE INVENTION

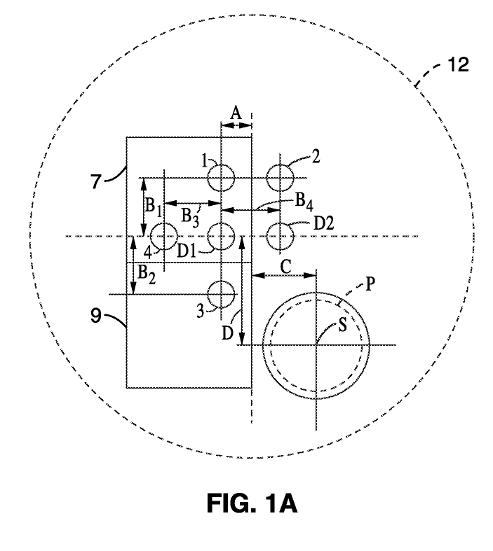

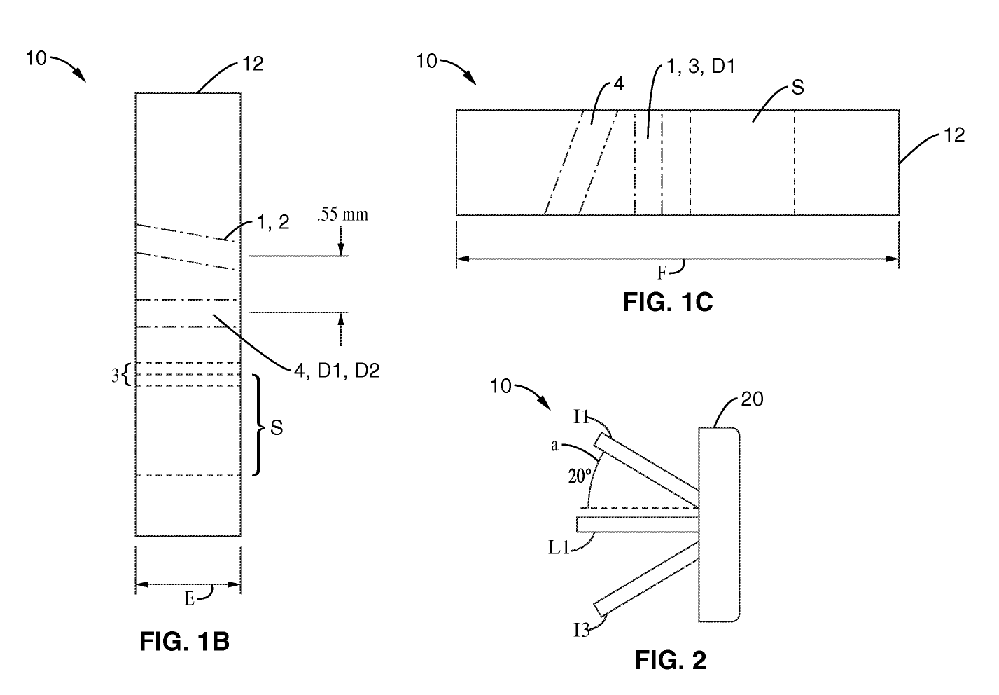

Referring more specifically to the drawings, for illustrative purposes the present invention is embodied in the apparatus generally shown in FIG. 1A through FIG. 17B. It will be appreciated that the apparatus may vary as to configuration and as to details of the parts, and that the method may vary as to the specific steps and sequence, without departing from the basic concepts as disclosed herein.

FIGS. 1A-3 show various aspects of a probe 10 according to one embodiment of the present disclosure as follows.

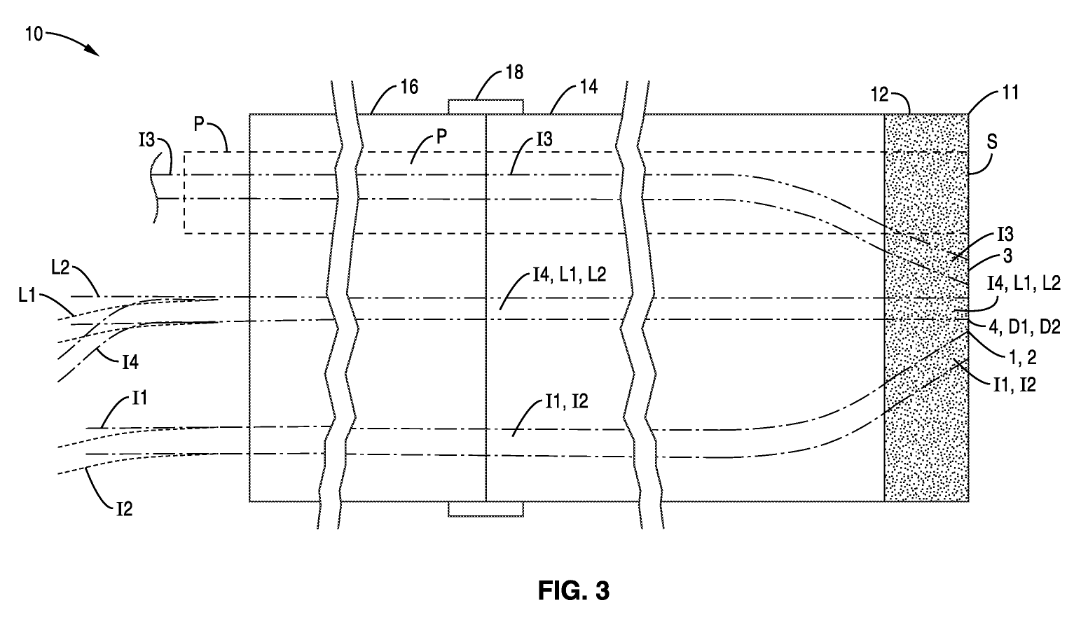

More specifically, FIGS. 1A-C show an end-view, side view, and bottom view, respectively, of a schematic layout of a disc housing 12 that is located at distal tip 11 of probe 10. It is to be appreciated that while certain ones of these Figures show certain internal features of the probe in shadow for further understanding, other internal features may not be shown in order to provide enhanced clarity of those particular internal features that are shown. Disk 12 provides a housing for various features contained therein as follows. Two holes D1,D2 for housing the distal ends of light delivery fibers L1,L2 (shown in FIG. 3), respectively, and four holes 1,2,3,4 for housing the distal ends of light collection or imaging fibers I1,I2,I3,I4 (also shown in FIG. 3), respectively, are arranged in a particular relative spatial arrangement and relative distances between them at the distal tip 11 of disk 12, as shown in end view in FIG. 1A. In addition, a hole S for housing a pressure sensor P (shown in FIG. 3) is also provided. For purpose of various aspects of the description of the embodiments elsewhere under the present disclosure, light collection or imaging fibers described may be given reference indicators I1,I2,I3,I4 to correspond with the respective light signal intensities measured, also referenced as I1,I2,I3,I4. Such collection fibers may also be represented by alternative designations 1,2,3,4, respectively, thus corresponding with their respective locations in similarly designated holes in the probe tip, as would be apparent to one of ordinary skill in context of a particular portion of the disclosure. Furthermore, while “image” may be used to describe such fibers, it is to be appreciated that this references capturing signals incident on the fiber for collection and processing within a diagnostic system. In this regard, the term “image” thus merely represents the light characteristics so gathered, and though actual “imaging” of tissue may not be actually performed in colloquial sense (e.g. for visual observation or recognition of structures etc.).

The relative dimensions for the various features shown in FIGS. 1A-C are provided as follows (in terms of “about” the dimensions given): probe tip 11 thickness E=1.0 mm (see FIG. 1B); probe housing 12 at probe tip 11 outer diameter F=4.2 mm (see FIG. 1C); hole S=1 mm (providing clearance for a 0.8 mm outer diameter pressure sensor P); holes 1,2,3,4,D1,D2=0.25 mm; and relative distances between various features as shown are A=0.275 mm, B=0.55 mm, C=0.61 mm, D=1.0 mm, where A, C, and D are measured from center of the disk forming tip 11.

A first polarizer 7 that polarizes light in one direction is positioned on tip 11 to cover light delivery fiber hole D1 and light collection fibers corresponding with holes 1,4. A second polarizer 9 that is oriented to polarize light in perpendicular orientation relative to first polarizer 7 is positioned on tip 11 over disk 12 to cover light collection fiber hole 3. Light delivery fiber hole D2 and light collection fiber hole 2 are left uncovered by polarizers 7,9, or any other polarizer. According to one particularly beneficial embodiment, polarizer 7 passes light that is polarized parallel to a line connecting fibers I4 and L1 (corresponding with holes 4 and D1 at the probe tip); whereas polarizer 9 passes light parallel to a line connecting fibers I3 and L1 (corresponding with holes 3 and D1 also at the probe tip). In one further aspect of this relationship, it is appreciated that the polarizers are polarized in perpendicular fashion relative to each other according to this particular beneficial embodiment. The polarized and unpolarized light corresponding with these various fibers provides certain particular benefits in analyzing the light scattering properties of certain tissues according to further embodiments of the present disclosure, as is elsewhere further described hereunder.

In addition, as shown in FIGS. 1B-C, the holes 1,2,3,4 are each angled, by about 20 degrees, to each converge toward a respective one of holes D1 and D2. This is in order for certain collection fibers housed in the respective holes to be angled relative to the respective light delivery fiber housed in the intended delivery holes D1,D2 that provide incident light into tissue for scattered collection by the respective collection fibers. More specifically to the specific angled arrangements shown in the Figures, as shown in the particular plane illustrated in FIG. 1B, holes 1,2 are angled relative to delivery fiber holes D1,D2 (which extend relatively straight or parallel through tip 11) with about 0.55 mm separation between the centers of the respective holes 1,2 and the center of holes D1,D2. As shown in FIG. 1C, in that relative plane perspective (which is perpendicular to the plane perspective showing angled holes 1,2), hole 4 is angled to converge toward hole D1.

As further shown in FIG. 2, light collection fibers I1,I3 converge at about 20 degree angles toward light delivery fiber L1 at the interface with tissue 20 at the probe tip (disk and probe tip not shown). Further aspects of this angled arrangement of light collection fibers relative to respective light delivery fibers, in context of illuminated tissue, are elsewhere herein described.

Further overall features of probe 10 are illustrated in the longitudinal side view shown in FIG. 3. Here, the arrangement of light delivery fibers L1,L2 housed within probe 10 and registered with holes D1,D2 are shown, as are light collection fibers I1,I2,I3,I4 relative to holes 1,2,3,4 at disk 12 at probe tip 11. In addition, pressure sensor P is shown in relative arrangement seated within hole S in the disk 12 at probe tip 11. These respective light and pressure members are also shown extending proximally through a housing body of probe 10, which also includes a distal outer member 14 and a proximal outer member 16.

Various materials and constructions for the probe 10 body components may be employed, as would be apparent to one of ordinary skill, so long as the respective light and pressure members are appropriately protected and maintained in relative arrangements in order for the probe 10 to function as intended. However, in one particularly beneficial construction, distal outer member 14 is a stainless steel hypotube, which in additional beneficial embodiments is filled or potted (such as for example with epoxy), and proximal outer member 16 may be for example a reinforced but more flexible tubing (vs. relatively stiff distal outer member 14). These respective outer member parts are bound together to form a robust joint and overall probe construction, such as for example by using shrink tubing capturing their abutting interface (as shown schematically at shrink tubing jacket 18 in FIG. 3). The relative lengths of the respective regions of the probe 10 may vary to suit a particular need or intended use within the broad aspects contemplated hereunder. However, in one particular beneficial mode constructed and used such as according to certain experiments performed, the distal end portion corresponding with distal outer member 14 may be about 6 inches long, with the proximal end portion about 12 feet long.

As shown schematically in FIG. 3, the respective light collection/imaging fibers I1,I2,I3,I4, respective light delivery fibers L1,L2, and pressure sensor P extend from the proximal end 13 of proximal outer member 16 of probe 10. This is generally accomplished with further outer protective jackets over each respective proximally extending member (e.g. fiber or sensor), which extends to a proximal coupler (not shown) to interface with respective component in an overall system for intended use.

Various specific materials and methods may be employed in order to manufacture a probe providing features and utility consistent with one or more of the broad aspects contemplated by the present disclosure. However, for purpose of further illustration, the disk 12 and incorporated light delivery and collection fibers, and pressure sensor, and overall probe 10, are manufactured as follows.

The fibers I1,I2,I3,I4,L1,L2 are glued into disk 12 in their respective holes 1,2,3,4,D1,D2 (such as variously shown in FIGS. 1A-C, and FIG. 3). A water soluble plastic fiber is inserted into the hole S for the pressure sensor P. The fibers are all polished flat to a suitable optical finish at the surface of disk 12. The polarizers 7,9 are then glued onto the end of the probe 10 in the appropriate location and orientation, respectively (such as shown in FIG. 1A). The outer diameter of the disk 12 is then machined down to the final diameter, which in the particular beneficial detailed embodiments is about 4.2 mm. The outer surface of the probe is then again polished to an optical surface with a 200 micron layer of epoxy over the polarizers. The water-soluble fiber is then dissolved from hole S and replaced by sensor P.

For purpose of providing still further understanding, examples of further detail related to the steps for manufacturing an exemplary probe as generally outlined above are provided as follows.

1. Layout 6 pieces of about 12 feet each of optical fiber, e.g. Fiberguide APC200/220/260N fiber.

2. Insert fibers into Multimode Fiber Optics Inc. SPM29 metal interior/plastic clad flexible tubing, with about 7 inches exposed on one end.

3. Cut the SPM29 tubing so about 12-15 inches is left on the other end.

4. File interior of SPM29 tubing so no rough edges exist that may cut the optical fibers.

5. Put about a 1.5 inch length of small shrink tubing over fibers on 7 inch end, but do not shrink yet.

6. Insert fibers through HTX-07T-06 body tubing and locating cylinder over fibers on 7″ end.

7. Insert polymer through 1 mm hole in carbon disc and into HTXX-17R_{—}06 sensor tubing.

8. Insert all fibers into carbon disc, be sure the angles are correct—the detection/collection fibers must point toward the delivery fibers. The more angled looking holes should be on the tubing side.

9. Slide collar over disc and press disc to lock. Slide tubing up until it mates up against disc.

10. Using Epotek 301 epoxy, glue probe face as follows. Tilting probe face down (e.g. about 30 degrees) insert epoxy down body of probe using care not to get epoxy into sensor tube. Let cure 24 hr. under the heat lamp about 12-16 inches from probe. Then reposition the probe face up (e.g. perpendicular to assembly table) and add a blob of epoxy on top of probe. Let cure 24-48 hours under heat lamp about 15 inches away from probe.

11. Polish probe face starting with a 12□m polishing disc. Polish until reaching the carbon disc.

12. “Soak out” polymer; advance a “poker” insert (e.g. a 0.39″ drill bit) to biopsy needle that is 1 mm in diameter through tubing. This may require gently hand drilling the hole. Water may be squirted (e.g. using a syringe) in the distal end of the sensor tubing to also help dissolve the polymer.

13. Using pads and diamond, polish starting at 2□m grit and progressing down to 0.25□m grit.

14. Dry thoroughly, insert new length of polymer so it sticks out further than the thickness of the polarizers but is several mm into the tubing.

15. Attach polarizers (e.g. 3M 37% polarizer film) to face of probe using care to align properly under a microscope. A pointed object, such as broken wooden stirrer because of the static of the polarizers, may be used. Tack down polarizers with “superglue” on a corner for each polarizer.

16. With the probe in a face up position, glue with Epotek 301 so that the surface is covered with a nice mound but the polymer is still poking out. Cure.

17. Contact machinist and get probe machined to final diameter.

18. Coat outside circumference of probe with epoxy, and cure.

19. Polish face of probe as in step 11, using care as to not polish off the polarizers.

20. Glue SMA connectors to ends of fibers, and polish.

21. Soak out polymer, insert pressure sensor (e.g. FISO Technologies fiber-optic sensor, FOP-MIV-NS-338C, 5 psig range), and connect electronics.

For still further illustration, more specific exemplary materials and methods related to preserving hole S and assembling sensor P into the probe are provided as follows.

The sacrificial fibers used to preserve hole S during various stages of the probe manufacturing operation are composed of poly(vinylpyrrolidone) (e.g. “PVP”). This is a water soluble polymer with thousands of known uses. Other water soluble polymers would also work for this application, however, including for example but without limitation: poly(vinylalcohol), poly(vinylcaprolactam), modified cellulosics, poly(ethylene glycol) (e.g. poly(oxyethylene)), and also including biopolymers such as for example but without limitation sodium alginate, guar gum, and xanthan gum.

PVP was chosen for use in typical physical embodiments manufactured and used to conduct experiments according to the present disclosed probes, and was considered particularly beneficial in terms of simplicity and availability. Water soluble polymers have been used as mold releases (as films or coatings). This present application is particularly well supported by a polymer fiber that has sufficient strength to be handled and provides a sacrificial barrier to the optical adhesive which is used in making the probe. A round cross-section assists the formation and maintenance of a continuous hole through the cured epoxy. Although several molecular weights have been used and observed to provide suitable results in some applications, such as with the preparation of clear singular fibers (drawn from a solution of chloroform), a particularly beneficial technique uses a cotton string which is dip-coated in 10% poly(vinylpyrrolidone) (USP-pharmaceutical grade K90) contained in ethanol solvent. The coating is sequentially made until thick enough for the application (e.g. about 1 mm diameter for use in a hole S of such diameter). This operation is similar for example to making a candle, except that the solvent is evaporated between dippings.

It is noted that the molecular weight of the polymer, however, may be varied, in particular with respect to PVP which is readily soluble in water. However, for one particular beneficial example, a suitable range may be between about 10,000 to about 3,600,000 D, with a particular beneficial embodiment observed at about 1,600,000 D.

Further to the methodology of manufacture, a PVP/cotton fiber (not shown) is fed through the 1 mm hole S in the probe tip disk 12. When the rather fluid epoxy mixture is placed on the probe tip 11, the PVP/cotton fiber (a composite) prevents epoxy from filling the hole. When the epoxy is cured, the PVP is simply dissolved in water, leaving a clean hole, and a continuous hole through the epoxy where the pressure detector fiber or pressure sensor P can then be inserted. The advantages of having the cotton string (fiber) inside include, without limitation: 1) allows water to wick into the center of the composite fiber, to help dissolution of the PVP from the inside (instead of just from the exposed ends), and 2) allows one to pull on the string to loosen the remaining material (allowing faster dissolution). The process is repeated in order to provide a clean resulting lumen for seating the pressure sensor S. The following references are herein incorporated in its entirety by reference thereto: “Water soluble polymers: solution properties and applications” Edited by Zahid Amjad, Plenum Press New York 1998, especially for example pp 259, (ISBN 0-306-45931-0); and a list of applications provided at http://www.ispcorp.com/products/perfchem/content/products/perchems/pvp.html. Certain exemplary physical properties in terms of K-Values, Ranges, and molecular weights are provided as follows (K-Value/Range/Molecular Weight): K-15/13-19/9,700; K-30/26-35/66,800; K-60/50-62/396,000; K-85/83-88/825,000; K-90/88-100/1,570,000; K-120/114-130/3,470,000.

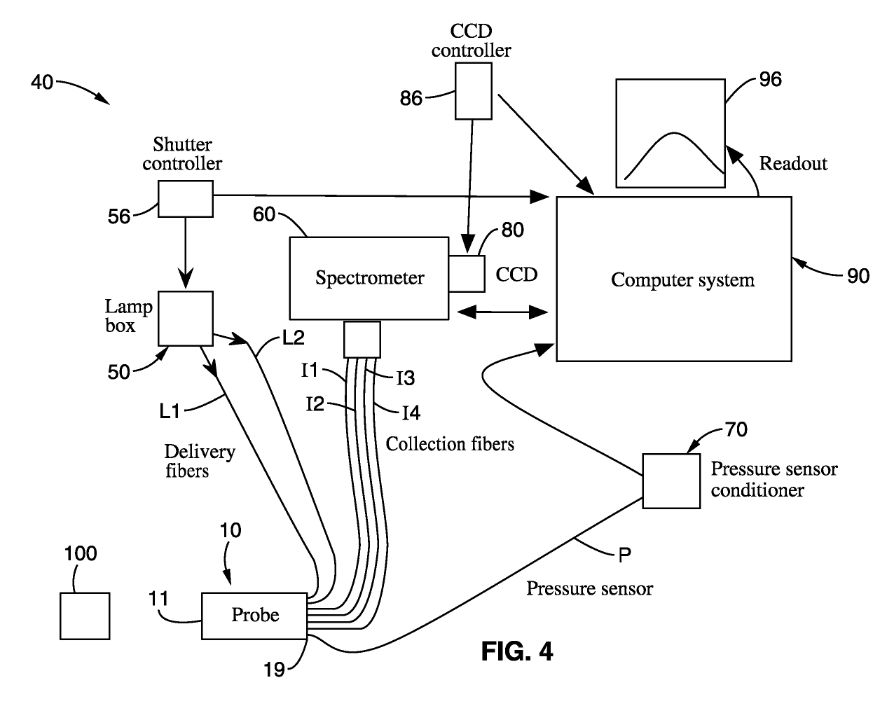

A probe 10 as just shown and described by reference to FIGS. 1A-3 is used within an overall system 40 as shown in FIG. 4. FIG. 4 shows system 40 to include probe 10, a lamp box 50, a spectrometer 60, a pressure sensor conditioner 70, a CCD 80, and a computer system 90.

More specifically, light delivery fibers L1,L2 are optically coupled from probe 10 to lamp box 50. Computer system 90 is coupled to a shutter controller 56 that controls a shutter mechanism (not shown) in lamp box 50 in order to illuminate the respective light delivery fibers L1,L2 at different respective times during a tissue analysis being performed. Within lamp box 50, two separate light sources may be included to respectively couple to light delivery fibers L1,L2. Light collection fibers I1,I2,I3,I4 are optically coupled from probe 10 to spectrometer 60 that is coupled to a CCD 80. Computer system 90 is coupled to a CCD controller 86 that controls CCD 80, and receives information related to light collected at respective fibers I1,I2,I3,I4 at different times and corresponding to tissue illumination via respective light delivery fibers L1,L2.

Computer system 90 includes software that processes information related to light delivered and collected, and performs certain calculations related to various parameters included in data analysis for the intended use of the system. In highly beneficial modes of operating the system 40 as just described, this analysis is performed in order to provide useful information in diagnosing presence or absence of certain tissue types in the analyzed object, such as in particular beneficial modes cervical cancer or pre-cancerous tissues in women. Computer system 90 will typically produce an output useful in performing such diagnosis or further analysis toward that end, such as readout 96 shown schematically to represent a graphical output of one or more useful parameters in performing such diagnosis or further analysis.

FIG. 4 also shows a reference material 100, which is provided for use with the system 40 according to one mode as follows. Reference material 100 is a material that reflects light in a reproducible manner. By measuring a spectrum of the reference material every time the system is used, or at regular intervals during prolonged use, variations of several parameters (e.g. lamp intensity), can be eliminated from the final data obtained from the system.

In one beneficial embodiment, the reference material 100 is titanium dioxide. In a still further beneficial embodiment, this is provided in a container (e.g. a glass jar with an opening just wide enough to insert the probe 10). The titanium dioxide material is provided in epoxy at the bottom of the container and covered by water. This reference material 100 provides for one or more baseline measurements to be taken by system 40 for ultimate use in processing data taken from tissue for diagnosis with the system 40, as described in finer detail elsewhere hereunder.

Various specific devices may be used for the components in the system 40 just described by reference to FIG. 4. However, for purpose of illustration, certain specific suitable examples are provided as follows.

Roper Scientific CCD Spec 10:400, 1340×400 pixels;

WINSPEC software;

Acton 3001 spectrometer;

Uniblitz VMM-D3 shutters and controller;

Hoya glass UV blocking filter, Y-48;

FISO Technologies fiber-optic pressure sensor, FOP-MIV-NS-338C;

FISO Technologies FTI-10 signal conditioner;

Windows computer; and

Gilway L1041 halogen lamps.

The components for probe 10 may include, for example: Fiberguide APC200/220/260N optical fibers for fibers I1,I2,I3,I4,L1,L2; HTX-07T-06 stainless steel tubing for distal outer member 14; HTXX-17R-06 stainless steel tubing to hold the pressure sensor P; Epotek 301 epoxy to fill the distal outer member 14; 3M 32% polarizer film for polarizers 7,9; Multimode Fiber Optics Inc. SPM29 metal interior/plastic clad flexible tubing for housing fibers I1,I2,I3,I4,L1,L2; and sacrificial polymer PVP90 coated fiber for filling hole S during other manufacturing operations prior to seating pressure sensor P in the assembly.

According to further embodiments, finer details of using the system 40 just shown and described by reference to FIG. 4, and using the novel probe 10 as also featured in that FIG. 4 and in further detail in FIGS. 1A-3, is provided as follows.

The two light delivery fibers L1,L2 are turned on and off sequentially. D2 delivers unpolarized light to the tissue. After entering the tissue some of the light is scattered and some is absorbed. Some of the scattered light will be incident on fiber I2 and collected to give an optical spectrum via spectrometer 60 and CCD 80 for computer system 90 to receive, store, and process. Light delivery fiber L1 delivers polarized light to the tissue. A polarizer 7 over delivery fiber L1 allows only linearly polarized light to pass and impinge on the tissue. The same polarizer 7 covers fibers I1 and I4, which collect light that is linearly polarized in the same direction as the incident light. A different polarizer 9 covers light collection fiber I3 so that it collects light which is polarized perpendicular to the polarization of the light incident through delivery fiber L1. All optical fibers used are generally chosen to be about 200 micron in diameter, with center-to-center separation between the delivery and respective collection fibers of about 550 micron. The numerical aperture of the fibers is about 0.37 according to the present particular embodiments. Fibers I1,I2,I3,I4 are angled 20 degrees toward the respective sources as illustrated for fibers I1 and I3 in FIG. 2. The purpose of the angle is to increase the sensitivity of the measurements to characteristics of epithelium. By placing the fibers at angle as shown and described for the present detailed embodiments, the scattered light collected by them represents a tissue penetration that is reduced so that more of the collected light is scattered from structures in the epithelium versus the deeper stroma.

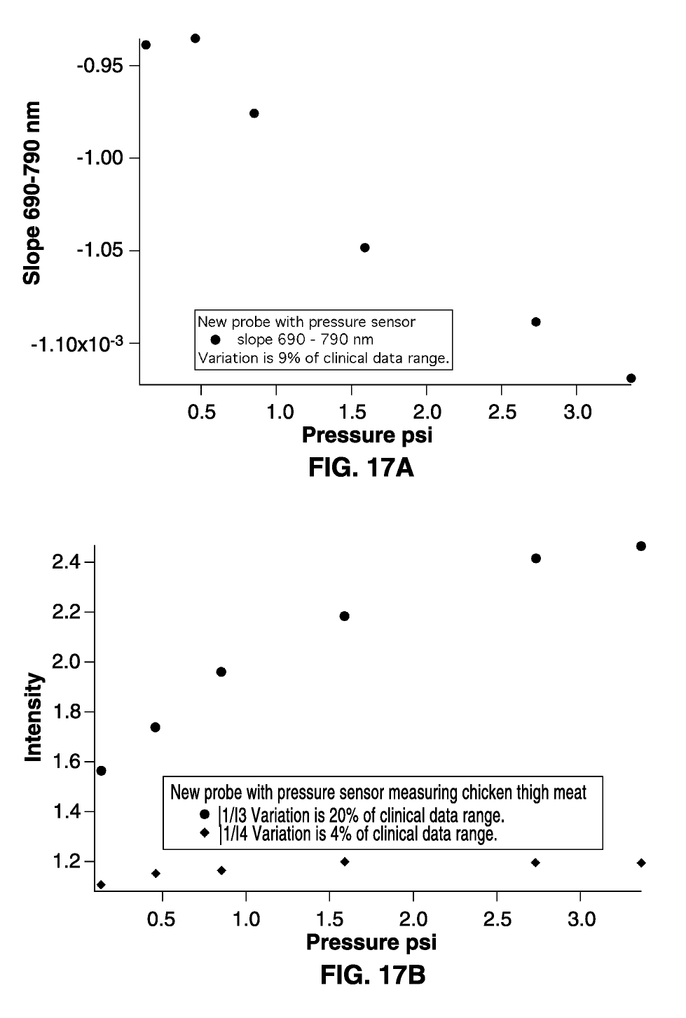

The ability of a similar fiber optic probe to provide information on the size and concentration of scattering centers has been demonstrated, without requiring angled fibers or pressure sensor. However, this was performed in aqueous medium and without pressure or pressure-sensitive measuring environs. Furthermore, a similar probe with angled light collection fibers, but without pressure sensing, was also used in in vivo clinical trials and shown to provide significant clinical diagnostic utility. One of the parameters determined to be useful for tissue diagnosis is total hemoglobin content. This parameter, however, depends strongly on the force to hold the probe against the tissue. As more force is used, the hemoglobin content appears lower. Accordingly, the particular useful parameter of hemoglobin content is compromised by variability, unpredictability, and lack of controllability presented by such probes without pressure sensitivity. Other aspects of measured parameters used in performing diagnosis have also been observed to vary over varied probe pressure against the tissue, such as for example with respect to varied scattering parameters over a range of applied probe pressure as shown in FIGS. 17A-B and described further below.

Accordingly, as contemplated by certain highly beneficial aspects of the present disclosure, this desirable additional benefit of enhancing such diagnostic systems with pressure sensitivity may be provided via a controllable pressure, or by a feedback control system that provides pressure monitoring and operating controls or data acquisition only at predetermined applied pressure ranges. In one particular beneficial present embodiment, a pressure sensor, as provided by probe 10 in FIGS. 1A-3 and incorporated into an overall system as in FIG. 4, allows the operator to know when they are in gentle contact with the tissue so they don't disturb the tissue morphology. This facilitates collection of quantitative information on the concentration of deoxygenated and oxygenated hemoglobin in the tissue, as calculated according to various more detailed embodiments presented hereunder, with variability due to pressure removed or at least significantly reduced for a more predictable and useful system and utility.

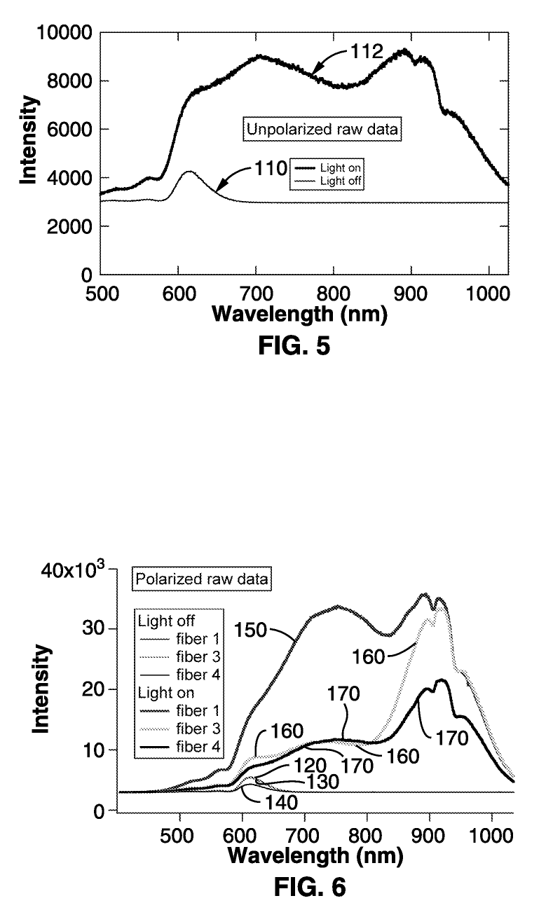

Examples of certain scattered light data acquired and processed according to the present embodiments, in particular with respect to using a probe 10, such as illustrated in FIGS. 1A-3, in a system 40, such as illustrated in FIG. 4, are provided for further illustration by reference to FIGS. 5-9 as follows.

More specifically, FIG. 5 shows spectra 110 and 112 for intensity over a range of wavelength taken at fiber I2 of a probe 10 against a tissue sample. These spectra 110,112 represent “light off” and “light on” conditions, respectively, for unpolarized light delivery fiber L2. The curves in FIG. 5 thus represent unpolarized raw data taken from a tissue sample. It is noted that the scale of the y-axis is detector-dependent, and as such may vary as between specific detectors that may be employed. Where “light on” and “light off” is indicated hereunder, it is to be appreciated by one of ordinary skill that a shutter over the source coupled to the respective light delivery fiber is open and closed, respectively.

FIG. 6 shows a graph of polarized light signal intensity scattered from the same tissue represented by the different signal represented in FIG. 5, also over a range of wavelength and according to a further mode of using a diagnostic system similar to that shown in FIG. 4. In contrast to the data in FIG. 5, this FIG. 6 represents data acquired during tissue illumination via polarized light delivery fiber L1 and as measured from three different light capture fibers I1,I3,I4 during on and off light delivery conditions in tissue. More specifically, curves 120,130,140 show signals measured by light collection fibers I1,I3,I4, respectively, during “light off” condition; whereas curves 150,160,170 show signals measured by light collection fibers I1,I3,I4, also respectively, during “light on” condition for tissue illumination via delivery fiber L1. These curves are provided in order to broadly represent exemplary data for the particular parameters which, as stated elsewhere hereunder, are measured in tissue to be diagnosed.

Light collection conditions at the light collection fibers is evaluated between “light on” and “light off” conditions, as shown in FIG. 5 (unpolarized) and FIG. 6 (polarized) because the “light off” condition gives measurement of the background light, and in this particular example, detector offset—these are artifacts desirably to be removed in the “light on” measurements taken for purpose of diagnosis. The “light off” spectra are subtracted from their corresponding “light on” spectra to provide an appropriately adjusted data set which is further processed in performing an ultimate diagnosis.

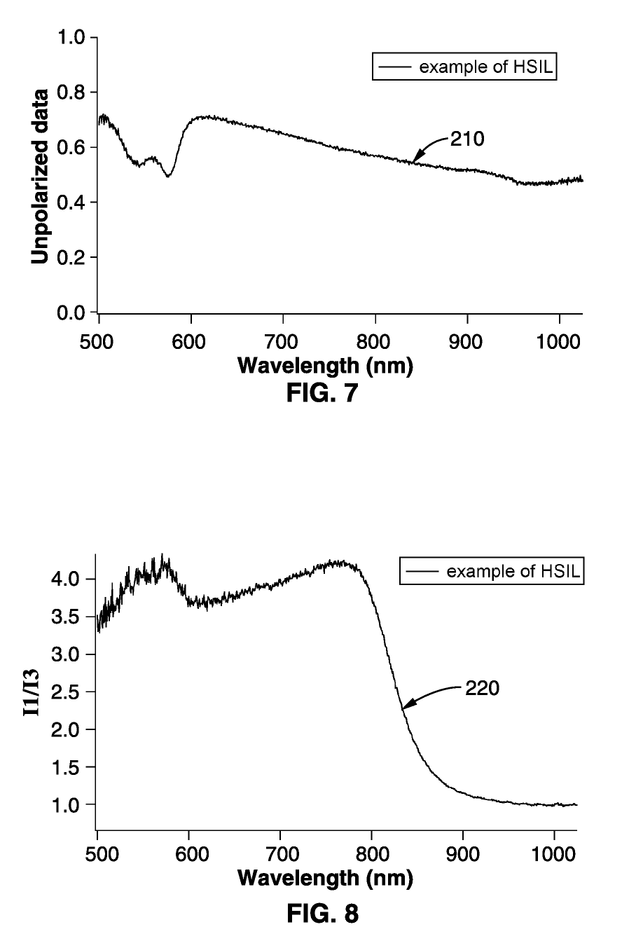

FIG. 7 shows another graph of unpolarized light signal intensity gathered following incident illumination of tissue over a range of wavelength, according to still another mode of using a diagnostic system similar to that shown in FIG. 4. This shows exemplary results indicating a presence of HSIL. The curve 210 shown in FIG. 7 for tissue representing the HSIL condition, was obtained by taking the type of unpolarized data shown in FIG. 5, subtracting the “light off” spectrum from the “light on” spectrum, and dividing the resultant spectrum by a “reference spectrum” which, though not shown hereunder, is described as follows. The “reference spectra” are obtained by placing the probe in contact with and perpendicular to a solid mix of titanium dioxide and epoxy covered in water, e.g. reference material 100 shown schematically in FIG. 4 and described elsewhere hereunder, acquiring spectra with the “light on” and the “light off” conditions for the unpolarized light from light delivery fiber L2, and then subtracting the “light off” spectrum from the “light on” spectrum.

It is to be appreciated according to the foregoing that slope along a resulting spectrum, such as represented in FIG. 7, is calculated over the wavelength range of about 690 to about 790 nm. As the curve graphed in FIG. 7 represents a ratio of underlying data, as described above, the y-axis is scaled to 1. This absolute value for the slope is generally greater for cancerous and pre-cancerous tissue than for tissue that is not cancerous or pre-cancerous. In this regard, to the extent the actual slope may be negative, this corresponds with a higher negative magnitude of actual slope (e.g. less than).

FIG. 8 graphically shows another spectrum via curve 220 that represents the ratio I1/I3 of light signal intensity reflected from tissue as measured at each of two light capture fibers I1 and I3 over a range of wavelength, according to still another mode of using a diagnostic system similar to that shown in FIG. 4. This also shows exemplary results with respect to this ratio indicating a presence of HSIL. This spectrum was obtained by taking the type of data shown in FIG. 6, subtracting the “light off” spectrum for fiber I1 from the “light on” spectrum for fiber I1, dividing this resultant spectrum by the reference spectrum for fiber I1, performing an analogous computation for the spectra for fiber I3, and then dividing the resulting data for fiber I1 by the resulting data for fiber I3. The spectrum is then normalized to an average value of 1 from between about 947 to about 997 nm. This is because the linearly polarized light in this wavelength range passes through the polarizer used in this embodiment equally well regardless of the relative orientation of the light polarization to the orientation of the polarizer.

According to the system and method ultimately represented in the calculated spectrum illustrated in FIG. 8, respective data acquired from non-cancerous and non-precancerous tissue has been observed to generally produce a lower value for I1/I3 over the data wavelength range from about 650 to about 750 nm than has been observed for cancerous and pre-cancerous tissue. Accordingly, data within this wavelength range that falls below a certain threshold is considered non-cancerous and non-precancerous, and data above the threshold is considered cancerous or pre-cancerous.

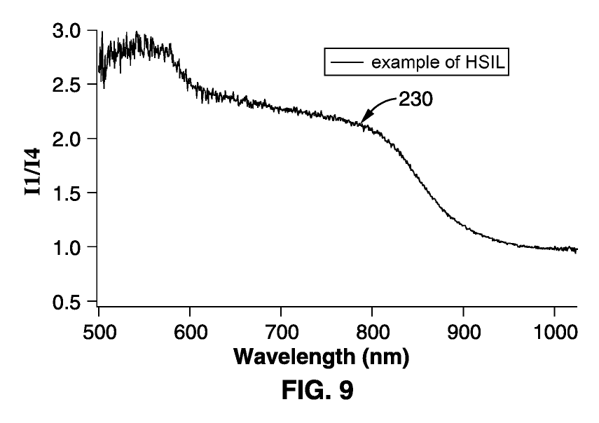

FIG. 9 graphically shows another spectrum 230 that represents the ratio I1/I4 of light signal intensity gathered from illuminated tissue as measured at each of light capture fibers 1 and 4 over a range of wavelength, according to still another mode of using a diagnostic system similar to that shown in FIG. 4. This also shows exemplary results indicating a presence of HSIL. This spectrum 230 is calculated in a similar manner to that just described above with respect to FIG. 8, except substituting the data acquired from fiber I4 in this present case of FIG. 9 to replace the fiber I3 data processed in the data represented in FIG. 8. Similar to the I1/I3 ratio comparisons noted above, data from non-cancerous and non-precancerous tissue has also been observed to generally produce a lower value for I1/I4 over the data wavelength range from about 650 to about 750 nm than has been observed for cancerous and pre-cancerous tissue.

Accordingly, as also noted above for I1/I3 ratio data, data within this wavelength range that falls below a certain threshold for I1/I4 is considered non-cancerous and non-precancerous, and data above the threshold is considered cancerous or pre-cancerous.

As is illustrated by the foregoing description by reference to FIGS. 5-9, substantial valuable data may be acquired by light scattering monitoring and analysis according to the present embodiments. However, it is appreciated that pressure remains a variable in systems not providing controlled pressure at the probe tip, or at least feedback and/or control in pressure monitoring and/or data acquisition within only particular pressure ranges. Various aspects of light-based diagnosis, and contribution of pressure enhancements, are provided by way of certain examples which follow immediately below.

### EXAMPLE 1

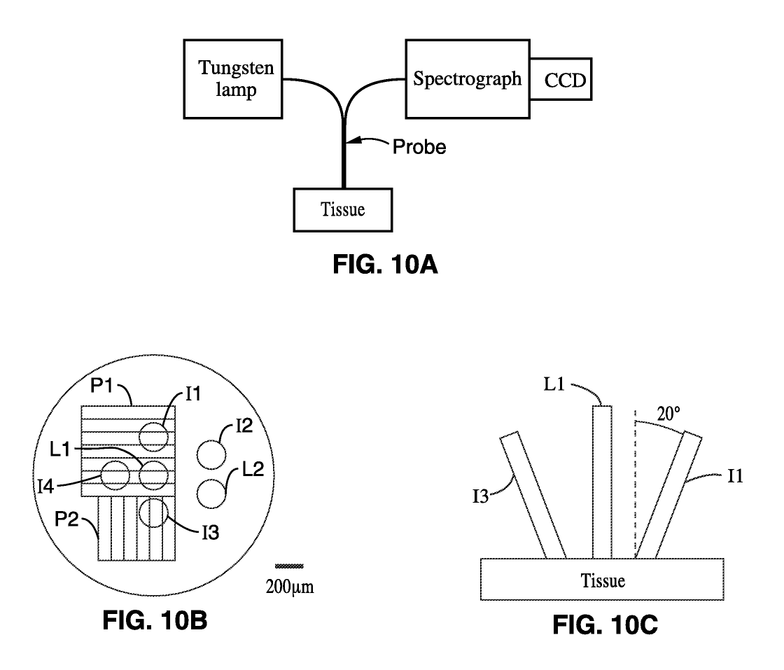

FIGS. 10A-14 variously relate to an experimental study performed as an example hereunder related to certain aspects of the present disclosure as follows.

The objective of the present experiment was to examine the utility of in vivo elastic light scattering measurements to diagnose high grade squamous interepithelial lesions (HSIL) of the cervix. A newly developed fiber optic probe according to various aspects of the present disclosure was used to measure light transport in the cervical epithelium of 36 patients undergoing standard colposcopy. Both unpolarized and polarized light transport were measured in the visible and near-infrared. Spectroscopic results of 29 patients were compared with histopathology of the measured sites using receiver operating characteristic (ROC) curves, multiple analysis of variance (MANOVA) and logistic regression. In analysis, three spectroscopic parameters are statistically different for HSIL compared with low-grade lesions and normal tissue. When these three spectroscopic parameters are combined, retrospective sensitivities and specificities for HSIL versus non-HSIL are about 100% and about 80%, respectively.

Accordingly, it is concluded that measurements of elastically scattered light show substantial benefit and utility as a non-invasive, real-time method to discriminate HSIL from other abnormalities and normal tissue. These results compare favorably with those obtained by fluorescence alone and by fluorescence combined with light scattering.

1. Introduction

The American Cancer Society estimates that in 2006, 9,710 cases of invasive cervical cancer will be diagnosed in the United States and 3,700 women will die from this disease. In the United States and western Europe, mortality from cervical carcinoma has significantly decreased coincident with the wide spread use of the Papanicolaou test (Pap smear) followed by colposcopy and detection of preinvasive and early stage disease. However, there are many limitations to currently acceptable screening and diagnostic strategies. From a clinical prospective, it is important to distinguish those pre-invasive lesions likely to progress to invasive carcinoma if left untreated in a cost-efficient manner. The Pap smear test frequently has a low sensitivity; high sensitivity and specificity are not achieved concurrently. Additionally, neither the Pap smear nor colposcopy-directed biopsy provide real-time diagnostic information. The patient must be contacted later to learn the results and set-up any future treatment/examinations. In the inner city clinics, up to 70% of patients with high grade lesions do not complete recommended follow-up examinations. “See and treat” protocols in which a Loop Electrosurgical Excision Procedure (LEEP) can be performed at the time of initial colposcopy have been proposed so that patients need not return for treatment. However inaccuracies of Pap smear results and colposcopic impression often lead to overtreatment. Improved diagnostics are necessary for “see and treat” protocols to reach their potential.

There are many methods under investigation to reduce screening and surveillance costs and improve detection of high grade squamous intraepithelial lesions (HSIL) which are a cervical cancer precursor. These include testing for human papilloma viruses (HPV) that are known to be associated with cervical cancer as well as several non-intrusive optical and optoelectronic methods. HPV tests have been shown to have high clinical sensitivity to HSIL; however, less than 10% of women with HPV have or will develop cervical intraepithelial neoplasia (CIN) III over a prospective 3 to 4 year time frame.

The basis for the optical techniques is to detect biochemical and morphological features that are concurrent with precancerous conditions. Examples of optical spectroscopy methods are elastic light scattering, fluorescence, optical coherence tomography and Raman spectroscopy. Fluorescence and Raman spectroscopy are primarily sensitive to biochemical changes, while light scattering and optical coherence tomography are primarily sensitive to morphological changes. The present disclosure provides results of polarized and unpolarized elastic light scattering measurements made using a wide range of wavelengths in the visible and near-infrared (NIR). These measurements provide useful information about both morphological properties and hemoglobin concentration and oxygenation.

The methods for measuring and analyzing light scattering in the current study were developed following results observed from previous light scattering measurements of tissue simulating materials (tissue phantoms). The intensity of detected light in a backscattering geometry such as that depicted in FIG. 10A depends on light scattering and absorption properties and is a function of wavelength. The only significant absorber in cervical tissue is hemoglobin which has strong absorption bands in the blue (e.g. about 420 nm) and green (e.g. about 540-580 nm) wavelength ranges. Between about 640 nm and about 900 nm the intensity of detected, unpolarized light depends only on the light scattering properties and is fairly featureless. The slope of unpolarized light intensity versus wavelength at wavelengths greater than about 600 nm has been observed to depend on proliferative status in previous measurements of mammalian cells. The slope has been observed to be steeper (and negative) for more rapidly proliferating cells than for quiescent cells. Additionally, this slope has been observed to depend on tumorigenicity. Consequently, the slope of unpolarized light intensity as a function of wavelength from 690 to 790 nm is one of the measured parameters in this study. Linearly polarized, elastic light scattering measurements provide additional information about morphological features. Specifically, measurements can be designed which are sensitive to the concentration and scattering strength of the scattering structures and to an effective average size of the scattering structures. These linearly polarized measurements have been observed to differentiate non-proliferating, non-tumorigenic cells from proliferating, tumorigenic cells.

The purpose of this study was to determine the sensitivity and specificity of in vivo light scattering measurements to discriminate HSIL from other lesions in patients undergoing standard colposcopic evaluation.

2. Materials and Methods

A. Spectroscopy

Optical spectra of cervical tissue were obtained in vivo with the fiber optic probe and experimental system shown in FIG. 10.

More specifically, FIG. 10 shows, in panel (a), a schematic of the measurement system. Light from tungsten lamps is delivered to the tissue by fiber optics. Light that has propagated through the tissue is collected by fiber optics dispersed by the spectrograph and incident on the CCD camera. FIG. 10(b) shows the distal end of the probe. The lines over fibers I1, I3, I4 and L1 indicate the direction of light polarization. Fibers L2 and I2 are unpolarized delivery and collection fibers, respectively. FIG. 10(c) provides a schematic showing the angle of some of the fibers with respect to the tissue.

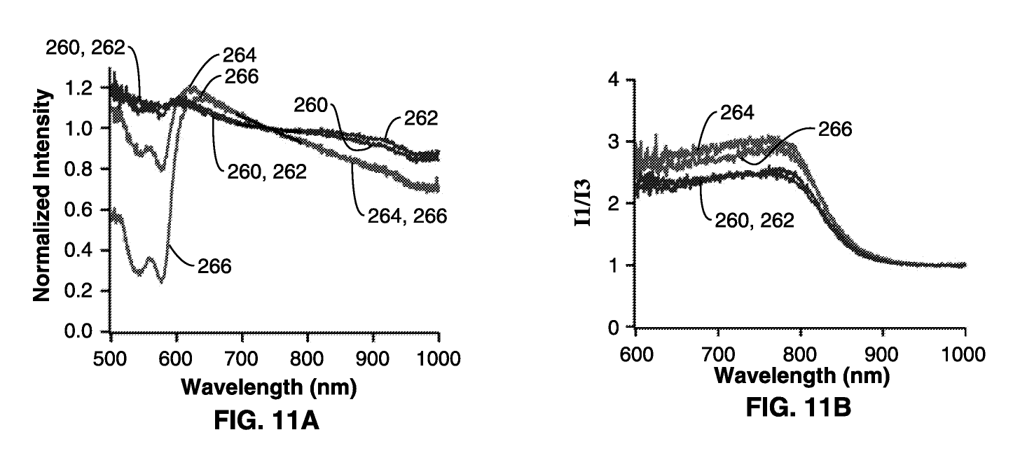

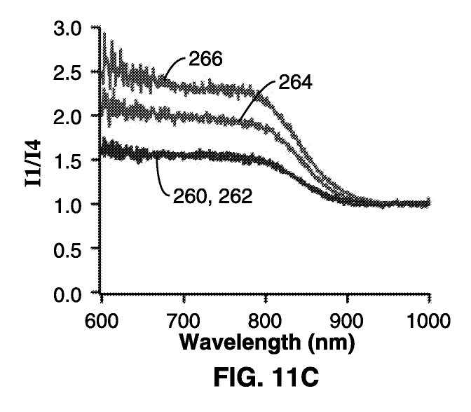

According to these features shown in FIGS. 10(a)-(c), in the experiment conducted the distal end of the probe was in contact with the tissue. Fibers L1 and L2 are polarized and unpolarized light delivery fibers, respectively. They are never on at the same time according to the specific operational modes used in the experiment. Fiber I2 collects scattered unpolarized light when L2 illuminates the tissue. Examples of unpolarized spectra are shown in FIG. 11a. The dips between 500 and 600 nm are due to hemoglobin absorption. Both the scattering and absorption properties of tissue are wavelength dependent.

Scattering properties also depend on the polarization of the light. Fibers I1, I3, and I4 are used to collect light when fiber L1 illuminates the tissue. A polarizer P1 over fiber L1 allows only linearly polarized light to pass and impinge on the tissue. The same polarizer covers fibers I1 and I4 which collect light that is linearly polarized in the same direction as the incident light. A different polarizer P2 covers fiber I3 so that it collects light which is polarized perpendicular to the polarization of the incident light. Examples of polarized data are shown in FIGS. 11(b) and 11(c).

All optical fibers used in the experiment were about 200 microns in diameter and the center-to-center separation between the delivery and collection fibers was about 550 microns. The numerical aperture of the fibers was about 0.37. Fibers I1, I2, I3 and I4 were angled at about 20° towards their respective sources as illustrated for fibers I1 and I3 by reference to light delivery fiber L1 in FIG. 10(c). The purpose of the angle is to increase the sensitivity of the measurements to characteristics of the epithelium. By placing the fibers at an angle, as conducted in the systems and methods of the present experiment, the penetration of the respective light scatter being collected is reduced so that more of the collected light scattered from structures in the epithelium rather than the stroma. Collection times ranged from 500 ms to 930 ms.

Accordingly, FIG. 11(a) shows certain unpolarized elastic light scattering measurements. FIG. 11(b) shows the ratio of polarized light intensities collected by fibers I1 and I3. FIG. 11(c) shows the ratio of polarized light intensities collected by fibers I1 and I4. In all graphs shown in FIG. 11, the two traces 264,266 are repeated measurement of the same site that was found by histopathology to be HSIL. The two traces 260,262 are repeated measurements of a site that was normal.

B. In Vivo Measurements and Pathology

The spectroscopic measurements were made after obtaining informed consent (under IRB approval from the University of New Mexico Health Sciences Center and Los Alamos National Laboratory). The data were taken during standard colposcopy procedures performed by one of four physicians. After 3% acetic acid was applied to the cervix, the optical probe was placed on sites that were to be biopsied and on additional sites the colposcopist interpreted as normal. All measurements were obtained in duplicate. Cervical tissue biopsies were obtained and placed in separate containers.

Thirty-six patients were evaluated. Three cases were not included in the analysis because the study pathologist did not have access to the biopsy samples or multiple samples were placed in the same container. Four cases were not used because of operator or spectroscopic equipment malfunction. For each patient used in the analysis, 2-4 sites were measured resulting in 88 total evaluable sites. All biopsies were examined by the same pathologist. The tissue was classified into five categories: normal, cervicitis (increased inflammation in the cervix mucosa involving predominantly the stroma but also in some cases the epithelium), low grade squamous intraepithelial lesion (LSIL), moderate HSIL (CIN II) and severe HSIL (CIN III). Biopsied tissue was also identified as ectocervix, endocervix or squamocolumnar junction (transformation zone of the cervix) (Table 1).

3. Data Analysis

Unpolarized data were normalized to an average value of 1 from 690 to 790 nm and the slope determined over this wavelength range. Example fits are shown in FIG. 11(a). The hemoglobin (Hb) concentration and ratio of oxygenated to deoxygenated hemoglobin were also estimated. The decrease in collected light intensity due to hemoglobin was assumed to be given by

$$I = {I}_{0} {e}^{- ( {C}_{Hb} {ɛ}_{Hb} + {C}_{Hb {0}_{2}} {ɛ}_{Hb {0}_{2}} ) L}$$

where C_{Hb} and C_{Hb0}_{₂} are the concentrations of deoxygenated and oxygenated hemoglobin respectively and ε_{Hb} and ε_{Hb0}_{₂} are the wavelength-dependent absorption of deoxygenated and oxygenated hemoglobin, respectively. L is the path length which was assumed to decrease weakly with increased hemoglobin concentration. The polarized data were normalized to an average value of 1 from between about 947 to about 997 nm. The polarizers do not work in this spectral range; therefore, this normalization corrects for the different light transport efficiencies of the optical paths. The ratio of intensities, I1/I4 and I1/I3 (where the ratios represent the respective light image fibers), were then calculated and examples are shown in FIGS. 11(b) and (c). The average values of these ratios from about 655 to about 755 nm were then determined and are referred to as ratio I1:I4 and ratio I1:I3.

In summary, the values of five variables were calculated from the spectroscopic measurements; slope, total hemoglobin, hemoglobin oxygenation, ratio₁₃ and ratio₁₄ (representing ratios of I1:I3 and I1:I4, respectively). Since two measurements were made of each site it was possible to determine the reproducibility of the measurements. For each site, an average and a difference of the two values were calculated for each variable.

Statistical methods used included a MANOVA, or multiple analysis of variance, to examine group mean differences among the outcomes of spectroscopic measurements. Groups were defined by pathological tissue diagnosis. Before use in MANOVA and multivariate logistic regression, the values of the five spectroscopic variables were each scaled so the range was 0 to 10.

Receiver operating characteristic (ROC) curves separating HSIL from LSIL, cervicitis, and normal were calculated for each spectroscopic variable. ROC curves calculated from the raw values show discrete steps. To obtain smooth ROC curves, the distributions of measurement values for the HSIL sites and the non-HSIL sites were fit to Gaussian distributions. These Gaussian distributions were then used to obtain smooth ROC curves.

Multivariate logistic regression was used to examine the predictive potential of the five metrics. One reason for choosing logistic regression, as opposed to other multivariate models was that the independent variables (i.e. the variables calculated from the spectroscopic measurements) do not have to be normally distributed, or of equal variance within each pathology group. The multivariate logistic regression equation is written as

$$P = \frac{1}{1 + {e}^{- ( {b}_{0} + {b}_{1} {X}_{1} + {b}_{2} {X}_{2} + {b}_{3} {X}_{3} + … )}}$$

where, P is the probability of the site being HSIL as a function of the spectroscopic metrics, X₁, X₂, X₃, . . . are measured spectroscopic metrics, and b1, b2, b3, . . . are results of the regression. For an increase in a measured spectroscopic variable, Xi of 10% of the total range of values for that variable, the increased risk of having HSIL is e^{b}^{ᵢ}. Significance is based on the Wald statistic, which is bᵢ divided by the standard error of bᵢ. An ROC curve was calculated from the probabilities calculated in the logistic regression analysis. The procedure is quite similar to that used to generate the other ROC curves. Sensitivity and specificity are calculated for a given cut-value and the cut-value was changed from 0 to 1 in steps of 0.01.

4. Results

Table 1 summarizes the number of sites measured for each pathology classification. Biopsies were obtained from thirty eight colposcopically abnormal sites. An additional 50 nonbiopsied sites were measured which were colposcopically normal.

According to the data reflected in Table 1 immediately above, the tissue locations are based on histopathology of the biopsies and were not recorded for 3 normal biopsied sites. Squamous epithelium is present in the ectocervix, columnar epithelium is present in the endocervix. SCJ is squamocolumnar junction and contains some combination of squamous, columnar and metaplastic epithelium.

MANOVA was performed to determine if the mean values of the spectroscopically determined variables differ between HSIL sites and all other sites. As shown in Table 2, the mean values of ratio₁₄, ratio₁₃ and slope are significantly different for HSIL sites than for non-HSIL sites when including colposcopically normal sites. A second MANOVA excluded the 50 colposcopically normal sites. As shown in Table 2, when only biopsied sites are considered, the mean values of ratio₁₄, slope and hemoglobin oxygenation are significantly different for HSIL sites than for non-HSIL sites. Ratiol3, which was significant when the colposcopically normal sites were included, is not significant. Univariate analyses verified the MANOVA results.

The overall significance of the data reflected in Table 2, as determined by either Wilks' lambda or Hotelling's trace, was <0.001 when colposcopically normal sites and biopsied sites were included, and 0.088 when only biopsied sites were considered. F-ratios were 5.3 for all colposcopically all sites and 2.1 for only the biopsied sites.

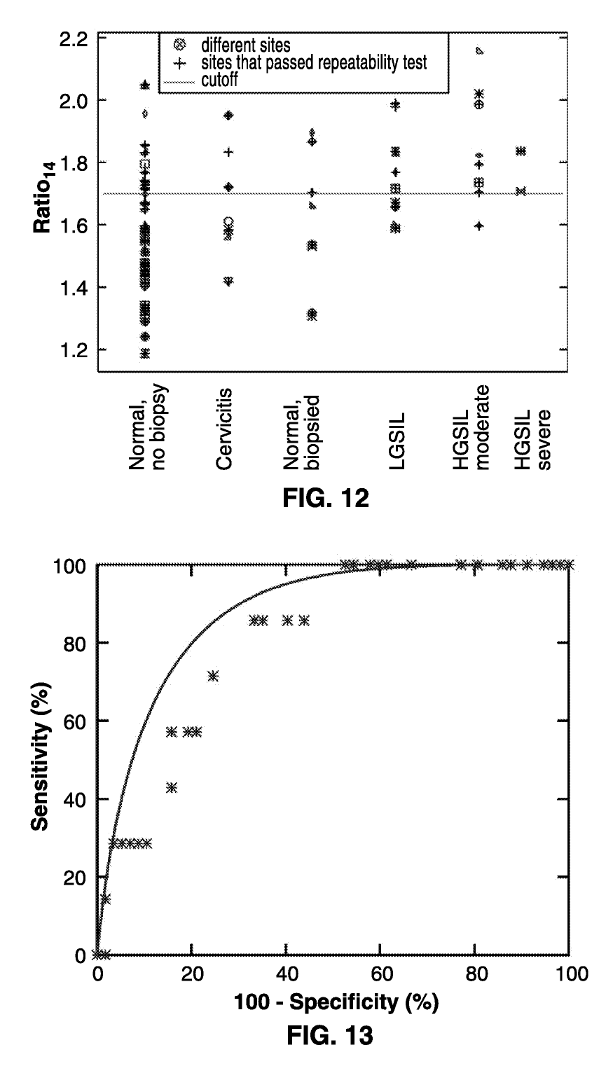

The values of ratio₁₄ as a function of pathology are shown in FIG. 12. Values of ratio₁₄ as a function of pathology. Certain symbols are the ratio₁₄ values with each patient represented by a different symbol. The 85% of the data that had the smallest difference in the repeated measurements of the same sites are marked with a “+” symbol. The average value for ratio₁₄ is shown for each site. When the 15% of the data with the largest differences in the values for the repeated measurements of each site are thrown out, only the values marked with a + remain. Assuming all values above a cut-off of 1.7 in FIG. 12 are HSIL and values below a cut-off of 1.7 are not HSIL gives a sensitivity of 86% and a specificity of 67% for detecting HSIL pathologies using only the points marked by the symbol “+”.

By moving the cut-off from a value of about 1.1 to a value of about 2.2, and calculating sensitivity and specificity at each cut off value, an ROC curve can be calculated. The star-shaped data points or symbols in FIG. 13 illustrate this ROC curve. A smooth ROC curve calculated from the same data is also shown. The ROC curve was calculated from measurements of ratio₁₄. The data points were calculated directly from the data in FIG. 12. The smooth curve was calculated assuming that the distributions of ratio₁₄ values can be described by a Gaussian for both the HSIL and non-HSIL sites.

The area under this curve shown in FIG. 13 is about 0.88. ROC curves for detection of HSIL versus all other pathologies were calculated for each of the other four spectroscopic variables. The ROC curves for total hemoglobin and hemoglobin oxygenation demonstrated that these variables did not have significant diagnostic potential.

Table 3 shows the areas under the ROC curves for slope, ratio₁₄ and ratio₁₃. The first column illustrates results obtained when the least repeatable 15% of the measurements are left out for each variable. The second column is the result when all data are used. The third column is the result when the normal sites that were not verified by biopsy are left out. The final column is the result when the same cut-off for reproducibility is used as for column one, but the colposcopically normal sites are not included.

In order to improve sensitivity and specificity, the diagnostic metrics were combined using two methods. For one method, classification as HSIL or non-HSIL was based on a majority classification by the metrics ratio₁₄, ratio₁₃ and slope. Each metric assigns each site to either the HSIL category or the non-HSIL category and the majority rules. In designing this metric, cut-off values for each metric to distinguish HSIL from non-HSIL were chosen such that a sensitivity of 100% was obtained. The corresponding specificity was 80%. Data from three sites for which the repeated measurements were very dissimilar were excluded (i.e. sites which were not part of the most repeatable 85% of the data for any spectroscopic variable).

Logistic regression was also used to combine the spectroscopic measurements into a single criteria for distinguishing HSIL from non-HSIL. Results of a multivariate logistic regression using slope and ratio₁₄ to “predict” HSIL are shown in Table 4. When the value of ratio₁₄ increases by 10% of the total range of ratio₁₄, the probability of the site being HSIL increases by a factor of 1.70. Similarly when the value of the slope changes by 10% of the total range of slope values, the probability of the site being HSIL increases by 1.84 when colposcopically normal sites are included and by 1.48 when only sites with biopsy confirmed pathology are considered. Further to the data reflected in Table 4, it is noted that three sites were excluded because repeated measurements of those sites gave very different results.

When ratio₁₃ is added to the logistic regression model, in an evaluation of only the biopsied sites, ratio₁₄ becomes significant at the 0.05 level as shown in Table 5. Several other logistic regression models were run and ratio₁₄ was the most consistent predictor. Some of the metrics appear to provide related information. For example, ratio₁₃ appears to provide information similar to that of slope, including it in the model reduces the significance of slope as shown in Table 5. Only biopsied sites were included in the Table 5 analysis. It is again noted that three sites were excluded because repeated measurements of those sites gave very different results.

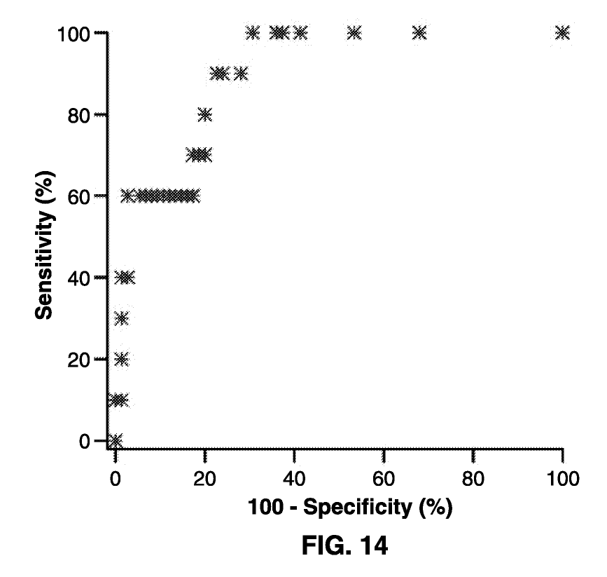

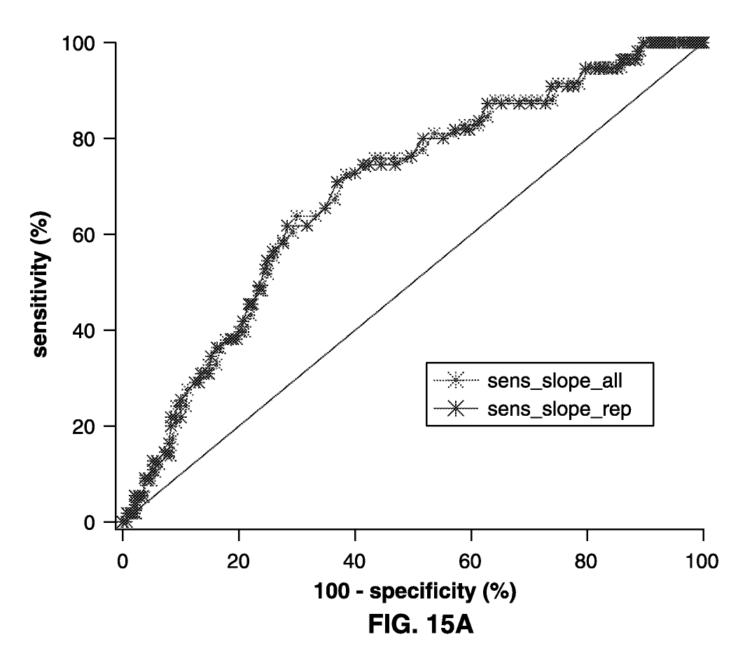

The ROC curve calculated from the posterior probabilities of a logistic regression analysis using ratio₁₄, ratio₁₃ and slope is shown in FIG. 14. The area under this ROC curve is 0.92, which is larger than any of the areas obtained when only using single metrics.

Morphology and consequently light transport properties of the cervix may depend on age, vaginal delivery or menopausal status. In this study, 44 sites were from patients who had not had a vaginal delivery, while 41 sites were from patients who had a vaginal delivery. A MANOVA analysis demonstrated that ratio₁₄ was significantly lower (p=0.006) for patients who had undergone vaginal delivery.

5. Discussion

Reported results of several clinical studies using fluorescence or light scattering/reflectance are summarized in Tables 6 and 7. More specifically, Table 6 summarizes results of point spectroscopic measurements; and results associated with the present experiment are provided in the bottom row of the Table 6. Sensitivity and specificity are on a per site basis. Certain of the abbreviations and notes used in the table represent the following: “sq.”=squamous; “fluor.”=fluorescence; “refl.”=reflectance; “LOO”=leave-one-out cross-validation; “colpo.”=colposcopy; ^{a}Squamous normal included acute inflammation and metaplasia. Table 7 summarizes imaging results, per in vivo, optical imaging studies of cervical pathologies. Sensitivity and specificity are on a per patient basis.

Associated sensitivity and specificities and even ROC curve areas (e.g. see Table 3) associated with this data from the present experiment and example are similar to previously published results.

Reported accuracies depend on the number of sites of different pathologies that are measured. For example, measuring sites that are clearly normal as was done in this study increases the specificity as determined on a per site basis because it is easier to differentiate HSIL from truly normal tissue than HSIL from metaplastic or inflamed tissue. Reported accuracies can also depend strongly on whether data are reported on a per site or per patient basis. In this present study, the specificity remains the same (e.g. about 100%) while the specificity drops from 80% to 55%, when calculated on a per patient basis. Reported accuracies may also vary depending on whether pathological verification was performed on all spectroscopic sites or whether colposcopic impression was also used as a gold standard as was done for many of the studies using stand-off imaging instrumentation.

When neural networks or other complicated multivariate algorithms are developed using training data sets, the algorithm may be trained to colposcopic impression rather than to actual pathology. Finally, the reported accuracies will depend on the statistical methods used. Ideally large and separate training and testing sets should be used. Unfortunately, large studies are extremely costly and time consuming and are therefore not ideal for testing of a new technique. Alternative strategies include leave-one-out cross validation which can overestimate the accuracy and/or reporting ROC curves. In conclusion, comparison of diagnostic accuracies reported in different studies is extremely difficult to do precisely.

In contrast to many prior studies, the spectroscopic parameters used in this study have physical interpretations. The spectroscopic parameters with the most significant p-values (see Table 2) were ratio₁₄, ratio₁₃, and slope all of which are related to changes in scattering properties. Ratio₁₄ and ratio₁₃ both increase when the effective size of the scattering centers decreases or when the number of scattering centers per volume increases. The slope becomes more negative when the effective size of the scattering centers decreases. The changes seen in ratio₁₄, ratio₁₃ and slope are all consistent with each other. Furthermore, these changes may be due to increased proliferative status of cells in dysplastic lesions. Finally, the epithelium has been shown to have increased scattering in CIN III which is also consistent with the changes seen in ratio₁₄, and ratio₁₃.

In our study, correlations were found between hemoglobin parameters and HSIL. Such correlation is believed to relate to angiogenesis in the stroma increasing with progression of dysplasia. Principal components derived from reflectance spectra which show the features of hemoglobin bands are useful for classification.

In addition to hemoglobin, other spectroscopic parameters used in the present experiment are also considered useful. For example, the slope of light intensity versus wavelength is observed to be diagnostically useful. In addition, the slope of a line that describes the wavelength dependence of the reduced scattering coefficient is also considered useful.

One advantage of using physically based measures rather than only a statistical analysis of the spectra is that the technique becomes less of a black box and the results may be presented to the medical staff in a more physiological/medically relevant manner. Providing a repeatable metric upon which to base diagnostic results, with low variability, is of particular value. An understanding of the relationship between tissue properties and the spectral measurements is valuable. In addition to precancerous and cancerous changes, other physiological changes may affect light scattering. Menopausal status affects the cervix and has been observed to alter its fluorescence properties. In this experiment, the ratio I1:I4 is observed to depend upon vaginal delivery status. Whether menopausal status affects light scattering measurements may be statistically confirmed by conducting further expanded study over an increased number of post-menopausal women than were observed in this study.

A particularly beneficial technique for identifying cervical dysplasias would cause minimal discomfort to the patient, be safe, be able to rapidly measure regions of interest in the visible cervix as well as in the endocervical canal, provide results in real-time, and detect all lesions with significant potential to become cancerous. The clinical data reported here regarding elastic light scattering and fluorescence measurements demonstrate that these optical techniques provide benefit. The techniques themselves do not cause any discomfort to the patient although all of the reported techniques were performed after application of acetic acid which can cause a mild stinging sensation in some women. The techniques require only low levels of light excitation, although care must be taken with UV excitation often used for fluorescence. The measurements are fairly quick, taking only a few seconds for point measurements and could provide results in real time. Before a technique is routinely applied in the clinic, refinements based on a detailed understanding of why the techniques sometimes fail are needed to improve the accuracy.

Prospective trials may be performed, following the results of this experiment, in order to further determine the accuracy by which tissue types of clinical interest, such as LSIL and HSIL, can be separated. With false negative rate kept low, ideally to approximately 0, as for the about 100% sensitivity in this study, with a reasonable specificity, then the devices and methods herein disclosed will facilitate increased use of LEEP or other treatments at the time of the diagnostic exam. Combined diagnostic and treatment appointments should benefit the patient by eliminating the need for another appointment (which is critical in poor and/or rural areas) and reducing the stress waiting for results. Combined diagnostic and treatment appointments should also reduce medical costs particularly if the initial equipment costs are low and if the costs of using the equipment are low compared to pathology. For further clinical utility, probes and systems, such as hereunder disclosed, may be further modified to also measure inside the cervical canal. In addition, a low cost system employing optical technology beneficially allows for use in areas were cytology/pathology are not readily available.

### EXAMPLE 2

An additional experimental study was performed according to certain additional aspects of the present disclosure, under the present example as follows.

1. Overview

The objective of the present experiment was to examine the utility of in vivo elastic light scattering measurements according to certain aspects of the present disclosure to diagnose high grade squamous interepithelial lesions (HSIL) and cancers in colposcopically abnormal regions of the cervix.

A fiber optic probe was used to measure light transport in the cervical epithelium of patients undergoing standard colposcopy. Spectroscopic results of 151 patients were compared with histopathology of the measured and biopsied sites. A method of classifying the measured sites into two clinically relevant categories was developed and tested using five-fold cross-validation. The effects of patient characteristics (e.g. age) on the spectroscopically measured parameters were also determined and used in developing the classification algorithm. Some spectroscopic parameters correlate with the status of a women's cycle, including both of (a) where she is in the cycle and (b) whether or not she has one. A spectroscopic variable known to correlate with how well tissue scatters light is also observed to correlate with age. Sensitivities in the low 80's and specificities in the 60's were obtained for separating HSIL and cancer from other pathologies and normal tissue. It is thus concluded according to this experiment that the sensitivity and specificity obtained in this study are very similar to sensitivities and specificities obtained in other large studies of optical diagnostics for the cervical dysplasia, provided hereunder however in a system and related method providing particular additional benefits over prior approaches.

The accuracy of the methods employed under this experiment is currently considered sufficient to provide clinical utility. However, other opportunities for improving upon the particular system and methods employed in this experiment remain. In many cases close collaboration between the pathologist and the spectroscopists, and a detailed understanding of the sources of light scattering, may be factors which can impact quality of results. Systems and methods, as elsewhere herein described, providing pressure sensitive techniques in use, are considered one particular example of such further improvements presented by certain further embodiments of this disclosure.

2. Methods

A. Instrumental

The experimental measurement system and probe are similar to those illustrated in FIG. 10 as described with respect to Example 1 above. Five parameters are obtained as follows: total Hb concentration, relative Hb oxygenation, slope of the scattering signal, and the rations of light collection fibers I1/I3 and I1/I4.

B. Clinical & Histopathology

Data from 151 patients have been acquired, and analyzed. Human subjects review boards reviewed and approved this work at respective institutions. Each patient was consented by the study coordinator.

Age, vaginal delivery status, menopausal status, cycle day (if nonmenopausal), and ethnicity were recorded for each patient. All tissue sites were measured once with the spectroscopic system and then the measurements were repeated. Subsequently biopsies were obtained. Biopsies were only taken if there was clinical necessity and each biopsy was placed in a separate container. Each biopsy was characterized as normal, cervicitis, LSIL, HSIL or cancer by the study pathologist. The study pathologist also ranked the inflammation as none, a few clusters of inflammatory cells, or many inflammatory cells. Vascularity was parameterized as normal or increased. The tissue site was determined by histopathology as ectocervix, endocervix or squamous columnar junction (SCJ).

C. Correcting Data for Small Differences Between Probes

Data from 64 of the patients were acquired from the original fiber optic probe dedicated to this study and data from the rest of the patients were acquired with a replacement fiber optic probe that was very similar, but not perfectly identical.

To determine if the change in probe had any effect on the measurements, data from the two probes were compared using students ttests within each pathology classification (except cancer). The mean value for I1/I4 was found to be different for every pathology classification. In contrast, no significant differences were found between the probes for I1/I3 for any pathology category. In summary I1/I4, water concentration, and total hemoglobin concentration were corrected for differences in the two probes. It is noted that the fine details of these probes were constructed for purpose of conducting the experiment under low level of control and without significant development for repeatability in manufacture. It is noted, as in standard course of any other medical device development, and in particular related to fiber optics, the probes may be made significantly more repeatable as a result of ordinary and customary device development targeting such repeatability.

D. Identifying and Correcting for Spectral Dependencies on Patient Characteristics

Several patient characteristics were correlated with the spectroscopic data; cycle day/menopausal status, age, and vaginal delivery status. To examine the effects of cycle day/menopausal status, the patients were first grouped into four categories; 1) have a menstrual cycle, 2) no menstrual cycle because of birth control, 3) pregnant or post partum and 4) menopausal. The Student's t-test was then used to determine if there were significant differences in the values of the spectroscopic variables between the different groups. P=0.01 was used to reduce type II error. Category 1 contained the largest number of measurements. Therefore, when differences were found between Category 1 and another category, the values in the other category were multiplied to make the average the same as for Category 1. After these corrections were made, there were no significant differences between categories. Category 1 was then divided into 3 subcategories; menstruating (days 1-6), fertile (days (7-20), and all other cycle days. These categories were compared using the Student's t-test and corrections were made when significant differences were found. The corrections were done in a manner that left the average for the original category of “have a menstrual cycle” unchanged.

To examine the effects of age, slopes of straight line fits to the spectroscopic variables versus age were determined. If the slopes were non-zero with a 95% or greater confidence then the effect was considered significant and the data were corrected so that the slope was one.

The Student's t-test was used to determine if there were any significant differences between sites in patients who had delivered a baby vaginally and those who had not. The comparisons were done within each diagnostic category. No significant differences were found.

E. Identifying and Correcting for Differences Between Doctors.

The Student's t-test was used to determine if there were significant differences in the values of the spectroscopic variables measured by the different doctors. The comparisons were done within each diagnostic category and P=0.01 was used to reduce type II error. Two doctors were found to have very similar data and when significant differences were found, the data were corrected to the average of the two similar doctors.

F. Probability Distributions

Histograms were made for each diagnostic category for each measured variable. These histograms were then fit to Gaussians and normalized to yield probability distributions.

G. Optimization of Classification Method

The optimization method for the classification method was very simple. A range of I1/I3 cut-off's was tried for set I1/I4 and slope cut-offs. The I1/I4 cut-off was then changed and the process repeated. Once a range of I1/I4 values had been tried, slope values were changed and the process repeated. The cut-off value for total Hb was not varied. A plot was made of all of the calculated sensitivity and specificity values. The optimum point was chosen to be the one with the largest sum of sensitivity and specificity with a sensitivity near or slightly greater than 80%.

H. Validation Method

Five-fold cross-validation was used as a validation method for the classification algorithms. The data were split into 5 subsets of approximately equal size with each subset containing approximately the same proportion of each pathology classification. Each of the 5 subsets were used once as a testing set with the remaining data used for training in each case. Sensitivity and specificity were estimated by averaging the results for the 5 data sets. This validation method was chosen because re-sampling methods, such as n-fold cross-validation, have been shown to be better at evaluating models than non re-sampling methods. Furthermore, leave one-out cross validation underestimates the errors of a model and 5-fold cross validation has been shown to be better for model evaluation.

3. Results

A. Pathologies, Epithelial Type, Inflammation and Vascularity

Table 8 summarizes the pathology of the measured sites as well as the epithelial type, amount of inflammation and amount of vascularity of the biopsied sites. The ectocervix is squamous epithelium, the endocervix is columnar epithelium and the squamocolumnar junction (SCJ) contains a combination of squamous, columnar and metaplastic epithelium. The vast majority of biopsied sites were of the squamous-columnar junction. On average, inflammation was increased for cervicitis, and HSIL as compared to the normal sites. Vascularity is more likely to be increased for cervicits and HSIL than in the normal and LSIL biopsies.

Column 1 in Table 8 shows the histopathology results of the present Example 2. Here, “normal” are non biopsied sites assumed to be normal by the colposcopist. The tissue locations, inflammation, and vascularity are also based on histopathology of the biopsies, and were not possible to determine for a few sites. Abbreviations used in the Table 8 include: “ecto”=ectocervix, “endo”=endocervix, and “SCJ”=squamous columnarjunction.

B. Dependence of Spectroscopic Parameters on Patient Characteristics

Menopausal Status and Cycle Day.

The values of I1/I4 and slope did not vary significantly between the categories of 1) have a menstrual cycle, 2) no menstrual cycle because of birth control, 3) pregnant or post partum and 4) menopausal. The average value of I1/I3 differed between categories 1 and 3, 1 and 4, 2 and 4, and 3 and 4. Significant differences were also found for totalHb, oxyHb and water. The data were corrected for all differences as described in the Methods section. Category 1 was divided into 3 sub-categories; menstruating (days 1-6), fertile (days 7-20), and all other days. The average of I1/I4 differed between the menstruating and fertile group, and between the menstruating and not menstruating group. The average value of slope differed between the fertile and not menstruating group. The average totalHb value differed between the menstruating and fertile group. The data were corrected for these differences.

Age

A fit of I1/I3 vs. age showed a decrease with age (95% confidence). All data were then corrected for the I1/I3 age dependence. None of the other spectroscopic variables had any significant dependence on age.

Vaginal Delivery Status.

No significant differences were found in the values of spectroscopic variables depending on vaginal delivery status.

C. Dependence of Spectroscopic Parameters on the Doctor.

A few differences were found between doctors, with the majority being between two particular doctors. The average values of I1/I3, slope, and oxyHb were all significantly different for these two doctors. Corrections to the data were made as described in the method section when significant differences were found.

D. ROC Curves

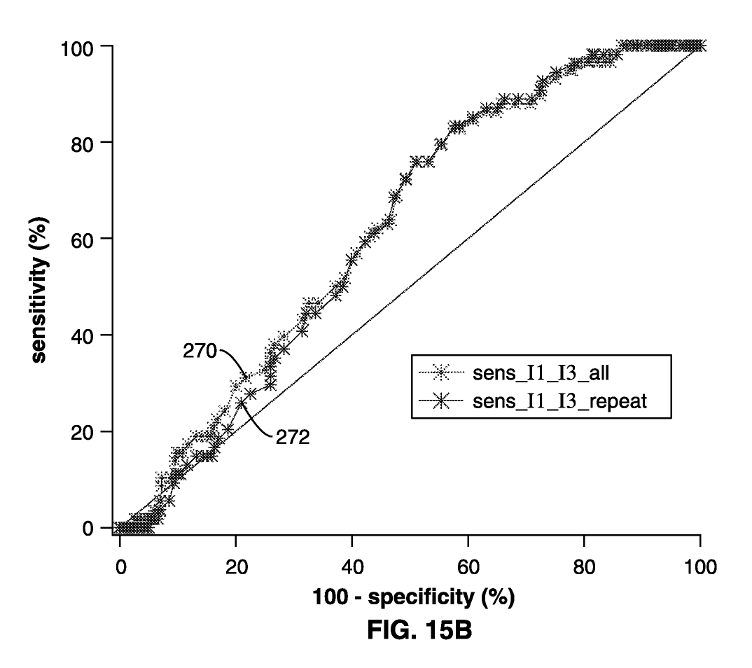

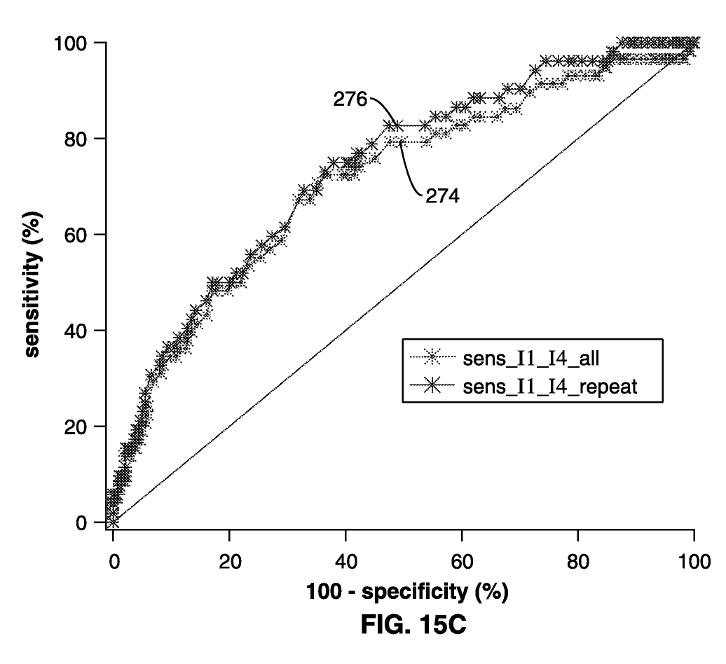

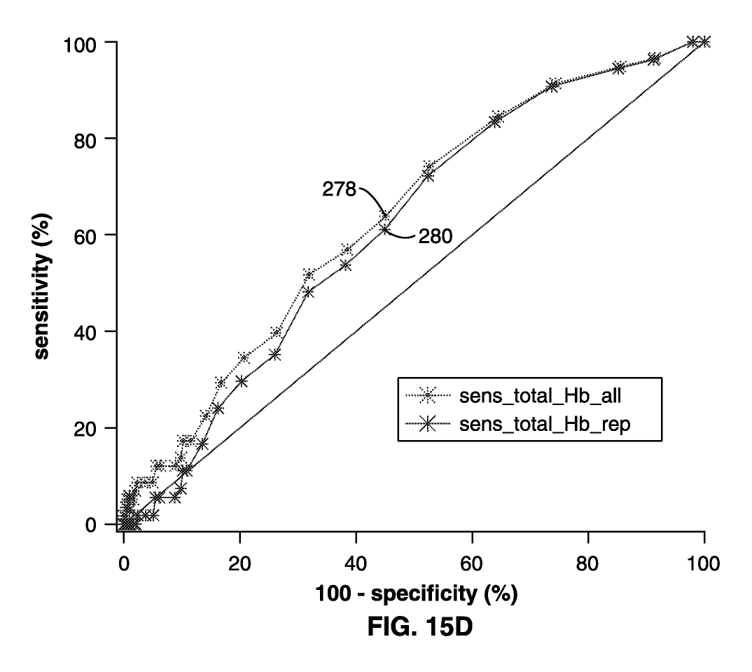

Receiver operating characteristic (ROC) curves for the diagnosis of HSIL and cancer versus the other pathologies are shown in FIG. 15, via various panels (a)-(d). For each variable two ROC's are shown. One curve on each graph shows the results when all data are used, such as for example at curves 270,274,278 in FIGS. 15B, 15C, and 15D, respectively. The second other curve shows the results when data that do not meet the reproducibility criteria for that variable are left out, such as for example at curves 272,276,280 in FIGS. 15B, 15C, and 15D, respectively.

It is noted that ROC curves are not shown for water and oxyHb, because the area under them was close to 0.5.

E. Probability Distributions

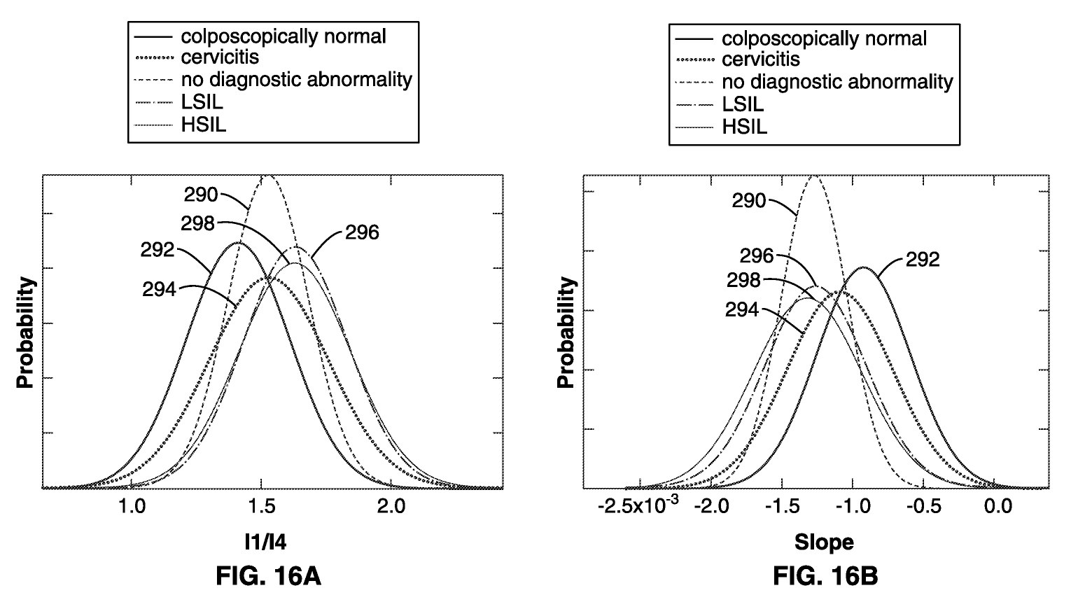

FIGS. 16A-B show the probability distributions of values of I1/I4, in FIG. 16A, and slope, in FIG. 16B, as obtained for the different diagnostic categories. The length of the x-axis is 1.4 times the range of measured values. More specifically, curve 290 represents no diagnostic abnormality, curve 292 represents colposcopically normal, curve 294 represents cervicitis, curve 296 represents LSIL, and curve 298 represents HSIL conditions, respectively.

Well separated probability distributions generally represent better separation of the diagnostic categories—e.g. ability to delineate between categories with the diagnosis performed. The best separation is observed between the categories of colposcopically normal and HSIL. LSIL and HSIL have very similar distributions. Biopsy normals are very different from “colposcopically normal” sites.

Narrow distributions allow for better separation of diagnostic categories. It is important to know whether the width of the distributions is due to biological diversity or instrumental variability. The distribution for “no diagnostic abnormality” is narrower than the other distributions. This indicates that some of the width of the other distributions is biological.

F. Diagnosing HSILs and Cancers

A primary objective of the present experiment is to characterize a system and method that separates (e.g. diagnostically distinguishes) HSIL's and cancers from other pathologies and normal tissue. The metrics with the most diagnostic potential are I1/I4, slope, I1/I3 and total hemoglobin content as shown by the ROC curves of FIG. 15. To diagnosis HSILs and cancers a method of combining these metrics was desired. In the course of analyzing the data several different methods were considered. The method chosen was a voting method. This method was chosen because of it's simplicity and the similarity between training and testing results. A vote is cast for each of three variables I1/I4, slope, and I1/I3. For each variable there is a cut-off value. If the measured value for a site is on one side of the cut-off then the vote is positive, i.e. for HSIL or cancer. If it is on the other side of the cut-off, it is for the negative category. The classification is then a two out of three vote. In other words, two out of three parameters agreeing results in a classification consistent with that agreement.

In addition, the voting method employed is qualified in that it provides totalHb with a particularly unique and strong weight in the analysis, providing quasi-“veto” power to the analysis. In other words, if totalHb is very high, it alone can change a classification of not cancer into cancer if there is a least one additional positive vote. The cut-off values were optimized as described in the materials and methods section. The results are shown in Table 9.

The best results are obtained with the positive category is HSIL or cancer and the negative category is non-dyslastic and when the colposcopy normal sites are included. The average results for the testing data sets are then a sensitivity of 80% and a specificity of 67%. When colposcopically normal sites are not included, the specificity drops to 48%.

Characteristics of the Incorrectly Classified Sites

The classification performed on the data set containing all of the biopsied sites (but no non-biopsied sites) was analyzed to determine if the miss-classified sites were of a particular epithelial type, had more or less inflammation, or had a more or less vascularity. The false positives did not have any tissue type dependence, or dependence on vascularity. Sites with no inflammation were slightly over represented in the false positive category.

Tissue from the squamous columnar junction was more likely to be classified as a false negative than were sites of pure squamous or columnar tissue. Tissue with a medium amount of inflammation was over represented in the false negative category, while tissue with a lot of inflammation was underrepresented. Sites with a high vascularity were underrepresented in the false negative category presumably because total Hb was used in the classification algorithm.

4. Discussion

A. Comparison to Other Studies

The number of patients in the study of the present Example, or n=151, is comparable to or significantly larger than that used in previously published studies. In comparing different studies, several details of the study must be considered. One is the resampling method. The leave-one-out method can overestimate the accuracy of a classification method. Also, the inclusion of sites that are expected to be normal may also bias a study results. According to the present experiment conducted under the present Example (e.g. as reflected in FIG. 16 and Table 9), separation of sites that appear normal by colposcopy from HSIL is more readily observed than separation of non-normal appearing sites from HSIL.

One previously published study reported results from 1569 measured sites in 271 patients. According to this study, both reflectance and fluorescence were measured and used in the analysis. The majority of the data were from biopsied sites that were colposcopically abnormal, however, if 2 biopsies were clinically necessary, then a study biopsy was taken at colposcopically normal location. Therefore, the results of this study are expected to give slightly better results than if only clinically needed biopsies were taken. One of the analysis methods used a random 70% of the data for training and the other 30% for testing and averaged the results from 100 repeats of this method. The sensitivity and specificity for diagnosing HSIL (vs. normal or non high-grade disease) on a per site basis were found to be approximately, 90% and 50%. These results appear slightly worse than the values of 80% sensitivity and 63% specificity according to the experiment of the present Example of this disclosure when all data are used.

Another previously published study reported results from 324 measured sites in 161 patients. Both fluorescence and reflectance were measured and the results were published separately. In addition to measuring up to two colposcopically abnormal sites, a colposcopically normal site of squamous epithelium was measured and biopsied, and if possible a colposcopically normal columnar site was also measured and biopsied. Therefore, the results from this study should be compared to results of the present experiment and Example of the present disclosure only to the extent the colposcopically normal sites are included. A leave-one-out resampling method was used which may increase the reported accuracy. Sensitivity and specificity on a per site basis using reflectance data for HSIL versus non-dysplastic squamous tissue were 72% and 81%, respectively. For columnar tissue, the analogous results are 72% and 83%. For fluorescence data, the squamous tissue results were 83% and, 80% while the columnar results were 72% and 78%. Since LSIL sites were not included in these results, they are comparable to bottom row of the left half of Table 9. The results under the present experimental Example of 80% specificity and 67% are of similar accuracy range. While the results of Mirabal et al. perhaps appear slightly better, it is believed that any differential is only due to the use of leave-one-out cross-validation method.

Results of one study of 111 patients have been previously reported on a per patient or per quadrant basis. Both fluorescence and reflectance data were analyzed according to this prior study. Further to this prior study, 28 women had cytologically and colposcopically normal cervices and 30 had non-neoplastic changes as identified by colposcopy. Therefore, this prior study contained a large number of cervices that would not be biopsied at colposcopy. Boot-strap resampling and five-fold cross-validation were both used to determine the accuracy of the model, and resulted in areas of 94.8% and 92.4% under the ROC curve as compared to the value of 94.7% obtained for non-resampled data. The optimized sensitivity and specificity for diagnosing HSIL versus normals and lesser pathologies were 95% and 83% on a per patient basis for non-resampled data.

Another study involving 97 test sites in 44 patients has also been previously conducted. According to this study, both light scattering and fluorescence data were obtained and used in the analysis. The resampling method was leave-one-out cross validation. There were 47 biopsied sites, while 50 of the sites were non-biopsied colposcopically normal sites. Sensitivity and specificity were computed for non-Sil sites vs. Sil sites. This separation differs from the other discussed studies in that the LSIL and HSIL sites were grouped together. There were only 2 LSIL sites as compared to 11 HSIL sites featured in this study. When the non-biopsied sites were included the sensitivity and specificity were 92% and 90%, respectively. When only the biopsied sites were included in the analysis, the sensitivity dropped to 71%.

Another study of 490 sites from 41 patients has also been previously published. Both diffuse-reflectance and fluorescence spectroscopy data were used in the analysis. 373 of the 490 measured sites were determined to be normal by colposcopic analysis. This is a very large number of normal sites and will greatly increase the accuracy of the results over a study using only sites normally biopsied during colposcopy. For resampling, the data were split into equal testing and training sets. This procedure was repeated more than a thousand times and the results averaged. Sensitivity and specificity for HSIL vs. (colposcopically) normal squamous tissue were 91% and 93% respectively for the fluorescence data. The analogous results for the diffuse-reflectance data were 82% and 67%.

In addition to the optical studies discussed above, a study using electrical impedance spectroscopy has also been published with 1168 measurements taken from 176 women. In this study, 680 measurements were of normal squamous tissue. The results are presented as areas under ROC curves. The area under the ROC curve for separating HSIL from mature metaplastic tissue was 0.89. The area under the ROC curve for separating HSIL from mature metaplastic tissue was 0.55. For comparison, the area under the I1/I4 ROC curve for separating HSIL and cancer from normals and other pathologies was 0.71.

B. Effects of Parameters Other than Dysplastic Status on the Spectroscopic Measurements

The values of the spectroscopic variables were found to correlate with characteristics of the patient, in particular (1) menopausal status and (2) cycle day as well as (3) age (even after correction for cycle day and menopausal effects). This information about how patient characteristics affect the spectroscopy data was used in the present Example in a manner that improved the quality of the data. Some of this information is routinely acquired in a clinical exam (e.g. age) and the other information can easily be acquired. In some cases, a patient does not know exactly when menstruation started. This is more likely to be the case later in the cycle when the exact day is less important.

It is noted that patient age was found not to affect the spectroscopic results in at least one previously published study. Accordingly, the present observations and use of age as a correction parameter in data analysis are considered of novel utility presented by the present disclosure.

The values of the spectroscopic variables also depended slightly on the doctor making the measurement. In the experiment according to the present Example, data analysis was corrected for these effects. However, this correction will be generally challenging to apply predictably and accurately in widespread uncontrolled use. These differences are believed to be caused by how the doctors hold and use the fiber optic probe. Certain modifications to the probe may be made that may alleviate or at least diminish the magnitude or prevalence of such user-dependent differences. One particular such modification, for example, includes providing pressure monitoring on the probe tip, and associated feedback control (either manual or automatic), to thereby provide a system that operates only within a predetermined range of applied probe pressure against tissue being evaluated. Such highly beneficial modification is provided elsewhere hereunder according to still further embodiments considered of particular benefit under this disclosure.

C. Clinical Utility

One objective of the present Example has been to provide and characterize a system and related methods that provide useful assistance to the colposcopist. The sensitivity of colposcopy is greater when two or more biopsies are taken. However, biopsies cause patient discomfort and increase costs. The system in its present configuration as used according to the present Example has a PPV of 53% and a NPV of 78% for HSIL's for non-dysplastic locations. Therefore, the system could be used to assist the colposcopist in deciding where to take a biopsy. Biopsies should be taken where the spectroscopic system gives positive readings, but not where the reading is negative.

In addition to other observations and comments provided hereunder, it is also noted that understanding whether the spreads are instrumental or biological may be of particular value in performing a proper analysis and diagnosis.

D. Other Comments

The data under the present experiment was also analyzed with the Mahalanobis distance metric, which is the analysis method used by Chang et al. and Mirabal et al. However, it was found that significantly worse results were obtained for the testing sets than the training sets, indicating that this method was over training. A “no vote” (meaning a determination of “not know” if cancer or non-cancer) category was preliminarily evaluated. While this approach to a voting method is considered a beneficial further embodiment of the present disclosure, in the particular setting of the data from this specific study enough improvement in accuracy to justify leaving out sites was not observed.

It is noted, and of particular benefit, that the measurement technique of this experimental Example is simpler than certain other methods previously disclosed. The present system and methods employed use only light scattering data, rather than light scattering and fluorescence used in combination in certain other prior efforts. The latter of these two techniques, fluorescence, requires a more sensitive detection system than is required in order to achieve suitably robust results according to the approach of the present embodiment of this Example.

5. Conclusions

Whereas the sensitivity and specificity of various diagnostic aspects of data obtained in this study may be similar to sensitivities and specificities obtained in other large studies of optical diagnostics for the cervical dysplasia, the present embodiments also provide further benefits over prior approaches. The accuracy of the methods demonstrated in the present embodiment is currently considered sufficient to provide clinical utility and benefit over previously disclosed systems and methods.

However, notwithstanding the distinct benefits of the current system and method featured in the present Example, opportunities to provide still further benefit via further improvements to these systems and methods remain. Certain improvements may be very difficult and require close collaboration between the pathologist and the spectroscopists and a detailed understanding of the sources of light scattering.

Other improvements, as presented elsewhere among the embodiments hereunder this disclosure, remove potential for artificial bias in the data due to user-dependent variables. One particular example of such improvement monitors probe pressure at the tissue interface where optical measurements are taken, in order to control measurements to particular pressure range representing only a particular degree of gentle forward contact. Such improvement provides relatively low cost, substantially user independent solutions that advance the systems and methods of the present Example forward toward still further clinical utility and benefit.

The following references are herein incorporated in their entirety by reference thereto:
L Breiman, P Spector, “Submodel selection and evaluation in regression—the X-random case”, International Statistical Review 60:291-319 (1992);
Mirabal Y N, Chang S K, Atkinson E N, Malpica A, Follen M, Richards-Kortum R. “Reflectance spectroscopy for in vivo detection of cervical precancer.” JOB 2002; 7:587-94;
Chang, S K, Mirabal, Y N, Atkinson, E N, Cox, D, Malpica, A, Follen, M, Richards-Kortum, R., “Combined reflectance and fluorescence spectroscopy for in vivo detection of cervical pre-cancer.” JOB 2005; 10; 024031-1,024031-11;
Georgakoudi I, Sheets E E, Mulller M G, Backman V, Crum C P, Badizadegan K, Dasari R R, Feld M S, “Trimodal spectroscopy for the detection and characterization of cervical pre-cancers in vivo”, Am J Obstet Gynecol, 2002; 186:3, 374-382;
Nordstrom R J, Burke L, Nile J A M, Myrtle J E F F, “Identification of cervical neoplasia (CIN) using UV-excited fluorescence and diffuse-reflectance tissue spectroscopy.” Lasers Surg. Med. 2001; 29:118-127;
Huh W K, Caster M R., Garcia F A, Gold M A, Guido R S, McIntyre-Selman K, Harper D, Burke L, Sum S T, Feeling F R O, Tavares R D, “Optical detection of high-grade cervical intraepithelial neoplasia in vivo: results of 604 patient study” Am. J Obit Gin 2004; 190:1249-57;
Ferris D G, Law head R A, Rickman E D, Holtzapple N, Miller J A, Grogan S, Bambot S, Agrawal A, Faupel M A, “Multimodal hyperspectral imaging for the noninvasive diagnosis of cervical neoplasia.” J. Lower Gen. Tract Disease 2001; 5:65-72;
J C Gage, V W Hanson, K Abbey, S Dippery, S Gardner, J Kubota, M Schiman, D Solomon, J Jeronimo, for The ASCUS LSIL Triage Study (ALTS) Group, “Number of Cervical Biopsies and Sensitivity of Colposcopy,” Obstetrics & Gynecology 2006; 108:264-272; and
Abdul S, Brown B H, Milnes P, Tidy J A, “The use of electrical impedance spectroscopy in the detection of cervical intraepithelial neoplasia,” Int. J. Gynecol Cancer, 2006; 16:1823-1832.

### EXAMPLE 3

As noted elsewhere hereunder, while highly beneficial light scattering measurements have been observed to provide significant utility in diagnosing presence or absence of certain abnormal tissue conditions, such as for example HSIL, pressure variability at the probe tip is believed to contribute significant variability and unpredictability, and thus marginalizing the utility, of such techniques. Accordingly, the present embodiments provide for novel inclusion of a pressure sensor associated with the probe tip in order to remove or at least significantly reduce this variability.

More specifically, it has been observed that increased pressure by a probe tip onto a tissue bed to be examined results in artificially biasing certain signal parameters, and thus compromising diagnosis. In particular, monitored light scattering parameters related to hemoglobin in the tissue appear to be biased by pressure, with increasing pressure reducing the observed hemoglobin signal. It is believed, and a premise to certain particular modes of various broad aspects contemplated hereunder, that providing slight gentle pressure against tissue provides optimal results. This ensures proper tissue contact and optimal tissue-device interface at the optical coupling from the probe into the tissue, while ensuring minimal bias from pressure to the signals of interest. The present embodiments provide a pressure sensor at the probe tip. This allows for monitoring the tip pressure against the tissue, so that it may be operated for data acquisition at optimal pressure.

Upon inserting pressure monitoring into the light scattering analysis provided by a diagnostic probe and system as provided according to the various embodiments hereunder, various different specific implementations may be arrived at for certain intended uses. For example, one predetermined pressure range representing robust yet gentle intimate contact with little to no pressure-induced biasing to tissue scattering properties may be incorporated into the system for pressure controlled light scattering monitoring. This threshold may be empirically determined based upon experimentation with a particular probe constructed for clinical use, and in reference tissue representing target tissue structures to be monitored. Such pressure range has been determined to provide particular beneficial results in evaluating tissue scattering properties of tissue, and which is believed to provide particular benefit for cervical cancer diagnosis. Such range may relate to all intended uses for the probe and diagnostic system, and which may span various types of tissues. Or, different ranges may be provided for different types of tissue, and which may be selected for example by a user in a user interface provided in the system (e.g. user inputs to the computer system controlling operation of the various system components). Furthermore, an initial “calibration” run may be conducted for a particular patient and tissue bed to be examined, such that optimal pressure range is defined based upon initial data acquisition taken and analyzed. For example, such a test run may involve gradually increasing pressure while illuminating the tissue and observing changes in signals received. Such monitored changes in light signals over range of increasing pressure may be used within the system to calibrate or “customize” the optimal range of pressure to take data for the intended tissue properties to be analyzed for diagnostic purposes.

The use of pressure monitoring to provide feedback used in running the light delivery and acquisition may be automated. For example, the computer system may operate the light delivery and acquisition components of the system only when monitored pressure is within a particular range. This may be done in “on/off” mode, operating when in range, and shutting off or blocking operation when not in range. Or, the light delivery and capture may be continuous over a period of time, but data acquired and used for signal processing and analysis may be only that data corresponding with pressure being within predetermined range of acceptability.

Alternatively, the system may provide indicia to a user that indicates whether pressure is within or out of optimal range in order to manually operate the data acquisition components appropriately. For example, a light indicator may indicate “on” (e.g. lit) when range is appropriate, and “off” (e.g. not lit) when pressure range is inappropriate or sub-optimal for robust data acquisition for intended analysis in the diagnosis. Or, these of course may be reversed. Providing still another alternative example, one light (e.g. a red light) may be lit to indicate pressure out of desired range, whereas another light (e.g. a green light) may be instead lit to indicate when pressure at the probe tip is in desired range.

Furthermore, it is to be appreciated within the broad intended scope of the present embodiments that a combination of manual controls and automated acquisition or control, e.g. via feedback control with certain manual components, may be employed.

Further aspects of the inclusion of pressure sensors and use of such information in an improved probe and diagnostic system are further developed as follows.

FIG. 17A shows a graph of certain data analyzed according to another experiment conducted under Example 3 hereunder using a probe and diagnostic system similar to that shown in various aspects in FIGS. 1A-4. More specifically, FIG. 17A compares slope of reflected light signal intensity along a particular range of wavelengths versus pressure of a monitoring probe against the tissue being monitored. As noted in the graph legend, the variation is 9% of clinical data range.

FIG. 17B shows a graph of certain other data analysis performed in the experiment conducted under Example 3. However, the current graph shows ratios I1/I3 and I1/I4 for reflected polarized light intensity taken at light capture fibers I1 and I3, and I1 and I4, respectively. This data is graphed over a range of probe pressure against the tissue being monitored, which in the current study was chicken thigh meat. As noted in the graph legend, the I1/I3 variation is 20% of clinical data range, whereas the I1/I4 variation is 4% of clinical data range.

Patent references and other documents herein described by reference to citations are all herein incorporated in their entirety by reference thereto, provided that to the extent their disclosure differs in conflict with certain aspects of the present disclosure bodily incorporated hereunder, the conflicting provision of the present disclosure shall prevail and the conflicting disclosure of the document incorporated by reference shall be considered background information for general background understanding of the art only.

In addition to other publications and information elsewhere herein cited, the following issued U.S. patents are also herein incorporated in their entirety by reference thereto: U.S. Pat. Nos. 5,303,026; 6,011,626; 6,381,018; and 6,639,674.

The following literature publications are also incorporated in their entirety by reference thereto:
Nath, A et al., “Effect of probe pressure on cervical fluorescence spectroscopy measurements,” Journal of Biomedical Optics, Vol. 9, No. 3, p 523-533 (May/June 2004);
Shim, M G et al., “In vivo Near-infrared Raman Spectroscopy: Demonstration of Feasibility During Clinical Gastrointestinal Endoscopy,” American Society for Photobiology 72, 146-150 (2000);
Mourant, J R et al., “In vivo light scattering measurements for detection of precancerous conditions of the cervix,” Gynecological Oncology 105 (2007) 439-445.
Mourant, J R et al., “Characterizing mammalian cells and cell phantoms by polarized backscattering fiber-optic measurements,” Applied Optics, Vol. 40, No. 28, p 5114-5123 (1 Oct. 2001);
Ramachandran, J et al., “Light scattering and microarchitectural differences between tumorigenic and non-tumorigenic cell models of tissue,” Optics Express, Vol. 15, No. 7, 4039-4053 (2 Apr. 2007); and
Nieman, L et al., “Optical sectioning using a fiber probe with an angled illumination-collection geometry: evaluation in engineered phantoms.” Applied Optics, Vol. 43, No. 6, p 1308-1319 (20 Feb. 2004);

According to the foregoing description, various broad aspects are contemplated notwithstanding the particular details of the particular embodiments variously representing such aspects (though such further details being considered of particular further benefit in the present description). For example, certain particular light parameters are monitored and analyzed in particular manners which provide useful and beneficial results. Certain particular arrangements between component parts are presented by the present disclosure, and which provide certain exemplary benefits according to the particular embodiments described in further detail. These arrangements are considered of independent novelty, benefit, and value, including in order to provide the particular applications and uses herein described with certain specificity, but also more broadly and as may be apparent in other applications or uses.

Various methodologies of data analysis and diagnosis are also herein disclosed, including for example with respect to certain correlations made between patient-dependent variables on diagnostic outcomes. For example, menopausal condition, menstrual cycle, vaginal vs. non-vaginal birth history, and age are certain such examples. These factors have been observed to present variables which correlate with outcomes in analyzing diagnostic data such as according to certain systems and methods presented hereunder. Accordingly, correcting or adjusting data analysis and outputs accounting for one or more patient specific characteristics, such as the exemplary factors just described, represents still a further aspect of the present disclosure of broad independent value and consideration. Though, it is again the case that such broad aspect, and related specific modes, are also of further value and consideration when further combined with other aspects and features of the embodiments set forth in certain detail hereunder.

In still another example, a voting method is described between multiple parameters monitored in order to produce a result. Various aspects are broadly contemplated, though illustrated by more detailed execution in the detailed embodiments featured in the disclosure. In one example, the tissue analysis performed provides an output categorization into one of three categories: (1) presence of abnormal condition (e.g. HSIL or cancer); (2) undetermined, or “not known”; and (3) absence of abnormal condition (e.g. HSIL or cancer). The voting method employed to execute on this broad aspect may include two or more of multiple measured parameters agreeing, or may require all agreeing. In certain settings for example, all parameters may be required to agree in order to produce a category “(3) absence of abnormal condition” conclusion. However, this may be varied according to further examples. Accordingly, for Z parameters, a category (1) result may be represented by >=A/Z parameters agreeing to presence of the condition, a category (2) result may be achieved by between >=B/Z disagreeing parameters as to presence or absence of the condition, and a category (3) result may be achieved by >=C/Z parameters agreeing to absence of the condition. Furthermore, notwithstanding such voting method, certain parameters, or value limits related to such measured or calculated parameters, may be weighted differently than others, and may in fact be given “veto” power to the voting system to alone definitively establish a categorization result. One such example presented in the detailed embodiments provides such weight to totalHb for example, if exceeding a particular level of detection.

Moreover, of additional particular note, incorporation of pressure sensors and pressure sensing in fiber optic probe tips has been hereunder presented by the present embodiments. Such pressure sensitive devices and systems, and use of pressure sensing in the methods, and furthermore correlations uncovered and used for accurate diagnosis, illustrate certain additional broad aspects of independent value and benefit when employed broadly together with measuring optical tissue properties according to the present disclosure. While considered broadly, such pressure sensing also provides further particular novelty and benefit in the various combinations disclosed in the detailed embodiments hereunder.

It is to be particularly appreciated, in addition to other aspects, one aspect of the present disclosure considered to provide especially beneficial use is an improved diagnostic system that detects abnormal physical properties of tissue in a patient based upon certain optical properties of the tissue. In one embodiment, a probe includes light delivery and capture fibers, and polarizers, to detect various optical properties in the tissue related to polarized and unpolarized light illuminating into, and then scattering from, the tissue. In one embodiment, the optical properties detected are processed and analyzed to produce results indicative of the physical property(s) being evaluated. The analysis corrects for certain physical characteristics of the patient as inputs to the system, such as menopausal or menstrual condition of women patients. Physical properties diagnosed include in particular cervical dysplasia conditions in women patients, such as HSIL, cervical cancer, LGSIL, or cervicitis. In various embodiments, analysis and diagnosis is based upon at least one of: ratios between certain scattered light signals captured from the tissue, slope of intensity over wavelength for certain scattered light signals captured, and hemoglobin-related parameters in the tissue. In a particularly beneficial further aspect providing significant improvement over prior approaches, pressure is monitored at the probe-tissue interface, with optical measurements taken and analyzed only at pressures falling within a predetermined range, generally representing gentle forward contact. In further embodiments, pressure monitored may provide a variable input into algorithmic calculations used for analyzing optical data acquired in performing a diagnosis. Moreover, in still further embodiments, pressure against tissue may be monitored in a calibration sequence that calibrates one or more pressure-dependent parameters for use in the data acquisition and analysis mode of operation. In various additional embodiments, output information provides indicia of one of three categories regarding a tissue condition being evaluated: (1) presence of the condition; (2) inconclusive; and (3) absence of the condition. In still further variations of these embodiments, the output information may be based upon one or more optical parameters acquired and analyzed, and may include voting methods between results taken from multiple parameters and/or calculations to yield an output result.

Although the description above contains many details, these should not be construed as limiting the scope of the invention but as merely providing illustrations of some of the presently preferred embodiments of this invention.

For example, it is to be appreciated that, while the particular features shown and described in FIGS. 1A-3 provide a probe with two alternatively illuminating delivery fibers interfacing with an external lamp box, and with multiple light capture fibers interfacing with an external spectroscope, alternative arrangements may be employed. For example, microcameras may be positioned at the probe tip, or otherwise within the probe, and then electrically coupled to an external source for processing of electrical signals without requiring optical transmission entirely along the probe and cabling to an external scope. In another example, light emission may be accomplished with emitters also at the probe tip, such as for example micro-laser diodes or light emitting diodes. In these regards, it is therefore to be appreciated that the particular optical fiber approaches of the detailed embodiments, though considered particularly beneficial, are illustrative of various devices or “members” that may more broadly either deliver or capture light, as the case may be, as intended by the overall devices and systems disclosed. In still another example, either two external light sources may be used, or one shuttered source, to illuminate two different light delivery fibers.

Furthermore, while particular coordinated arrangements between light illumination via certain designated light delivery fibers and light capture via other designated light capture fibers are herein described, other combinations may be employed for additional benefit diagnostic use. For example, it is to be appreciated that light capture fibers I1, I3, and I4 may be coordinated to capture light through the respective polarizer coverings during illumination of light delivery fiber L2 for subsequent analysis and diagnostic use of data related to optical properties of the tissue under that tissue illumination and capture environment.

Various aspects of the present disclosure relate to measuring certain parameters related to hemoglobin in tissue. Involved in certain embodiments are total hemoglobin, oxygenated hemoglobin, and/or deoxygenated hemoglobin. According to specific embodiments, the parameters analyzed include such aspects multiplied by the average distance light travels through the tissue. According to still further embodiments, actual oxygenation of the tissue may also be measured for analysis and processing for purpose of producing diagnostically useful results.”

Moreover, it is to be appreciated that such alternative approaches for illuminating tissue and capturing scattered signals therefrom are not all inclusive, and various combinations of such approaches, or still other alternative approaches, may be employed by one of ordinary skill without departing from the broad intended scope according to various aspects of the present disclosure.

Therefore, it will be appreciated that the scope of the present invention fully encompasses other embodiments which may become obvious to those skilled in the art, and that the scope of the present invention is accordingly to be limited by nothing other than the appended claims ultimately granted hereunder, and in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE 1
Characteristics of the measured sites

| Pathology | Number of sites | Ectocervix | Endocervix | SCJ |
|---|---|---|---|---|
| Assumed normal, | 50 | — | — | — |
| no biopsy | | | | |
| Normal | 9 | 1 | 1 | 4 |
| Cervicitis | 8 | 0 | 1 | 7 |
| LSIL | 10 | 2 | 0 | 8 |
| HSIL (moderate) | 9 | 2 | 0 | 7 |
| HSIL (severe) | 2 | 0 | 0 | 2 |

TABLE 2
MANOVA results. Three sites were excluded because repeated measurements of
those sites gave very different results

| | All sites | | | Biopsied sites | | |
|---|---|---|---|---|---|---|
| | | Mean ± SD | | | Mean ± SD | |
| Spectroscopic | | Non- | | | Non- | |
| variable | p-value | HSIL | HSIL | p-value | HSIL | HSIL |
| Ratio₁₄ | <0.001 | 4.5 ± 1.9 | 6.8 ± 1.7 | 0.030 | 5.4 ± 1.7 | 6.8 ± 1.7 |
| Ratio₁₃ | 0.002 | 4.0 ± 2.0 | 6.1 ± 1.9 | 0.091 | 4.9 ± 1.7 | 6.1 ± 1.9 |
| Slope | 0.001 | 4.3 ± 2.0 | 6.1 ± 1.9 | 0.048 | 4.9 ± 1.7 | 6.1 ± 1.9 |
| Total hemoglobin (Hb) | 0.106 | 0.9 ± 1.4 | 1.8 ± 2.9 | 0.273 | 1.0 ± 1.3 | 1.8 ± 2.9 |
| Hb oxygenation | 0.168 | 5.1 ± 2.2 | 6.1 ± 2.0 | 0.044 | 4.5 ± 2.1 | 6.1 ± 2.0 |

TABLE 3
Areas under ROC curves

| | Most | | | |
|---|---|---|---|---|
| Metric | repeatable 85% | All data | Only biopsies | Biopsied, repeatable |
| Ratio₁₄ | 0.88 | 0.78 | 0.67 | 0.69 |
| Slope | 0.85 | 0.85 | 0.72 | 0.77 |
| Ratio₁₃ | 0.82 | 0.74 | 0.60 | 0.67 |

TABLE 4
Results of a logistic regression analysis using ratio₁₄ and slope

| | All sites | | | Biopsied sites | | |
|---|---|---|---|---|---|---|
| | | Wald | | | Wald | |
| Variable | e^{b} | statistic | Significance | e^{b} | statistic | Significance |
| Ratio₁₄ | 1.70 | 6.07 | 0.014 | 1.68 | 3.61 | 0.077 |
| Slope | 1.84 | 6.90 | 0.009 | 1.48 | 3.14 | 0.057 |

TABLE 5
Results of a logistic regression analysis using ratio₁₄,
slope and ratio₁₃

| Variable | e^{b} | Wald statistic | Significance |
|---|---|---|---|
| Ratio₁₄ | 1.93 | 4.26 | 0.039 |
| Slope | 1.26 | 0.90 | 0.344 |
| Ratio₁₃ | 1.49 | 1.70 | 0.192 |

TABLE 6
In vivo, point optical studies of cervical pathologies

| Pathology stratification | Method | Validation | Sensitivity | Specificity | Patients |
|---|---|---|---|---|---|
| HSIL vs. non-HSIL | Fluorescence | Testing set | 79% | 78% | 95 |
| HSIL vs. sq. normal* | Fluorescence | LOO | 83% | 80% | 161 |
| HSIL vs. sq. normal* | Reflectance | LOO | 72% | 81% | 161 |
| HSIL vs. non-HSIL | Fluor. and refl. | LOO | 92% | 71% | 44 |
| HSIL vs. non-HSIL | Fluor. and refl. | LOO | 92% | 90% | 44 |
| included colpo. normals | | | | | |
| HSIL vs. non-HSIL | Reflectance | None | 100% | 80% | 29 |
| included colpo. normals | | | | | |

TABLE 7
In vivo, optical imaging studies of cervical pathologies. Sensitivity and
specificity are on a per patient basis

| Pathology stratification | Method | Validation | Sensitivity | Specificity | Patients |
|---|---|---|---|---|---|
| HSIL vs. sq. normal^{a} | Fluorescence | Testing sets | 91% | 93% | 41 |
| HSIL vs. metaplasia | Fluorescence | Testing sets | 90% | 87% | 41 |
| HSIL vs. sq. normal^{a} | Reflectance | Testing sets | 82% | 67% | 41 |
| HSIL vs. metaplasia | Reflectance | Testing sets | 77% | 76% | 41 |
| HSIL vs. non-HSIL | Fluor. and refl. | Testing set | ROC area: 0.8 | | 271 |
| HSIL vs. non-HSIL^{b} | Fluor. and refl. | Testing sets | ROC area: 0.92 | | 111 |

Abbreviations:
sq. squamous:
fluor, fluorescence:
refl, reflectance.
^{a}Squamous normal was determined by colposcopy and pathology.
^{b}Diagnoses were determined by either pathology or colposcopy.

TABLE 8
Characteristics of the measured sites.

| | | tissue location | | | inflammation | | | vascularity | |
|---|---|---|---|---|---|---|---|---|---|
| Pathology | sites | ecto | endo | SCJ | none | a little | a lot | normal | increased |
| “normal” | 181 | — | — | — | — | — | — | — | — |
| Normal | 36 | 12 | 2 | 20 | 19 | 9 | 5 | 33 | 0 |
| Cervicitis | 44 | 2 | 5 | 35 | 0 | 10 | 33 | 29 | 13 |
| LSIL | 43 | 6 | 1 | 36 | 10 | 12 | 17 | 31 | 6 |
| HSIL | 56 | 4 | 2 | 50 | 7 | 16 | 29 | 35 | 14 |
| Cancer | 2 | 0 | 1 | 0 | 0 | 0 | 2 | 0 | 1 |
| total | 362 | 24 | 11 | 141 | 26 | 47 | 86 | 128 | 34 |

TABLE 9
HSIL and cancer versus normal and all other pathologies

| | | | | | colposcopically | | | |
|---|---|---|---|---|---|---|---|---|
| | all measured sites | | | | abnormal sites | | | |
| | training | | | | training | | | |
| | set | | testing set | | set | | testing set | |
| | Sens. | Spec. | Sens. | Spec. | Sens. | Spec. | Sens. | Spec. |
| HSIL and | 84.8 | 64.4 | 79.6 | 62.6 | 82.3 | 45.7 | 83.2 | 44.8 |
| cancer vs | | | | | | | | |
| LSIL and | | | | | | | | |
| non-dysplastic | | | | | | | | |
| HSIL/cancer vs | 82.3 | 68.7 | 79.6 | 67.1 | 81.1 | 48.4 | 79.6 | 47.6 |
| non-dysplastic | | | | | | | | |

What is claimed is:

1. A medical diagnostic system, comprising:
a probe with a proximal end portion and a distal end portion with a tissue interface region;
at least one light delivery member coupled to the tissue interface region;
at least one light capture member coupled to the tissue interface region; and
a pressure sensor coupled to the tissue interface region.

2. The system of claim 1, further comprising:
a light illumination system coupled to the at least one light delivery member;
an optical measurement system coupled to the at least one light capture member; and
a pressure monitoring system coupled to the pressure sensor;
wherein the system is adjustable between an “off” mode and an operating mode when the tissue interface region is in contact with a region of tissue of a patient; and
wherein in at least the operating mode the light illumination system illuminates the at least one light delivery member to emit at least one incident light signal from the tissue interface region into the region of tissue, the optical measurement system measures at least one property of at least one captured light signal collected from the region of tissue by the at least one light capture member at the tissue interface region, and the pressure monitoring system monitors a pressure at the tissue interface region in contact with the region of tissue.

3. The system of claim 2, further comprising:
a controller coupled to the pressure monitoring system;
wherein the controller controls (a) at least one aspect of the operating mode of the system, and/or (b) an indicator that provides pressure-dependent indicia to a user useful in controlling at least one aspect of the operating mode of the system, based upon the pressure measured by the pressure monitoring system.

4. The system of claim 3, further comprising:
an algorithm accessed by the controller that determines a result based upon the measured pressure;
wherein the result is used by the controller to control the operating mode of the system or to provide the pressure-dependent indicia to the user in order to manually control the operating mode of the system.

5. The system of claim 4:
wherein the algorithm comprises a pre-determined pressure criteria associated with a binary “on-off” decision, such that a measured pressure meeting the criteria is associated with one of an “on” or “off” decision, and a measured pressure not meeting the criteria is associated with the other of the “on” or “off” decision;
wherein according to an “on” decision by the algorithm, the controller actuates the system into the operating mode or actuates the indicator to provide indicia to a user to adjust the system into the operating mode; and
wherein according to an “off” decision by the algorithm, the controller either does not actuate the system into the operating mode or controls the indicator to indicate to the user the “off” decision not to adjust the system to the operating mode.

6. The system of claim 5:
wherein the pressure criteria comprises a first pressure threshold;
wherein a measured pressure above the first pressure threshold corresponds with an “on” decision by the algorithm; and
wherein a measured pressure below the first pressure threshold corresponds with an “off” decision by the algorithm.

7. The system of claim 5:
wherein the pressure criteria comprises a first pressure threshold;
wherein a measured pressure below the first pressure threshold corresponds with an “on” decision by the algorithm; and
wherein a measured pressure above the first pressure threshold corresponds with an “off” decision by the algorithm.

8. The system of claim 7:
wherein the pressure criteria also comprises a second pressure threshold;
wherein the second pressure threshold is lower than the first pressure threshold, such that a pressure range criteria is provided between the first and second pressure thresholds;
wherein a measured pressure within the pressure range criteria corresponds with an “on” decision by the algorithm; and
wherein a measured pressure outside of the pressure range criteria corresponds with an “off” decision by the algorithm.

9. The system of claim 8, wherein the second pressure threshold is greater than zero and less than about 1 psi.

10. The system of claim 9, wherein the second pressure threshold is between about 0.5 psi and about 1 psi.

11. The system of claim 8, wherein the first pressure threshold is equal to or less than about 3 psi.

12. The system of claim 11, wherein the first pressure threshold is equal to or less than about 2 psi.

13. The system of claim 12, wherein the first pressure threshold is equal to or less than about 1 psi.

14. The system of claim 3, further comprising:
a processor coupled to the controller and also to the optical measurement system;
wherein in the operating mode the processor processes information corresponding with the at least one property measured by the optical measurement system and produces a result that is useful to a user in performing a medical diagnosis on the patient.

15. The system of claim 14:
wherein the information is related to presence of cancerous or pre-cancerous tissue in the region of tissue; and
wherein the result is useful to a user in diagnosing the presence of cancerous or pre-cancerous tissue in the region of tissue.

16. The system of claim 15:
wherein the probe comprises a cervical probe;
wherein the information is related to presence of cervical cancer or cervical pre-cancerous tissue in the region of tissue; and
wherein the result is useful to a user in diagnosing the presence of cervical cancer or cervical pre-cancerous tissue in the region of tissue.

17. The system of claim 14:
wherein the information is related to presence of HSIL in the region of tissue; and
wherein the result is useful to a user in diagnosing the presence of HSIL in the region of tissue.

18. The system of claim 3, further comprising:
an indicator coupled to and controlled by the controller so as to provide an indication useful to a user in controlling the operating mode of the system based upon the measure pressure.

19. The system of claim 18, wherein the indicator comprises at least one of a visual indicator and a sound indicator.

20. The system of claim 3, further comprising:
a first light delivery member with a distal end comprising a first light emitter at the tissue interface region and optically coupled to the light illumination system;
a second light delivery member with a distal end comprising a second light emitter at the tissue interface region and optically coupled to the light illumination system;
a first light capture member with a distal end comprising a first light collector at the tissue interface region and optically coupled to the optical measurement system; and
a second light capture member with a distal end comprising a second light collector at the tissue interface region and optically coupled to the optical measurement system;
wherein the controller in the operating mode controls the system such that the light illumination system illuminates the first light delivery member and second light delivery member independently during first and second unique time sequences, respectively, and such that the optical measurement system is controlled to acquire data from the first and second light capture members independently and uniquely during the unique time sequences, also respectively, corresponding with illumination of the respective light delivery members.

21. The system of claim 20, further comprising:
a third light capture member with a distal end comprising a third light collector at the tissue interface region and optically coupled to the optical measurement system;
a fourth light capture member with a distal end comprising a fourth light collector at the tissue interface region and optically coupled to the optical measurement system;
a first polarizer located over the first light delivery member and the first and fourth light capture members at the tissue interface region; and
a second polarizer located over the third light capture member at the tissue interface region;
wherein the second light delivery member and second light capture member are each uncovered and unpolarized at the tissue interface region;
wherein the second polarizer has a different polarization than the first polarizer; and
wherein the controller in the operating mode controls the optical measurement system and processor to recognize unique light signal data captured at the first, third and fourth light capture members during illumination of a respective light delivery member.

22. The system of claim 21, wherein the system comprises:
a processor coupled to the optical measurement system and controlled by the controller to process optical information in a manner that produces a diagnostically useful result based at least in part upon at least one of a ratio of light captured at the first and third light capture members, a ratio of light captured at the first and fourth light capture members, slope of at least one captured light signal or ratio over wavelength, total hemoglobin, total oxygenated hemoglobin, total deoxygenated hemoglobin, average distance light traveled through tissue, at least one further ratio of one or more of the foregoing against reference calibration light measurements taken with the probe, a difference between “light on” and “light off” operating conditions of one or more of the foregoing, and combinations thereof.

23. The system of claim 22, wherein the processor is configured to process the information and produce the result in a variable manner based upon at least one patient history or patient health parameter input.

24. A method for diagnosing a property of a region of tissue in a patient, comprising:
placing a tissue interface region of a probe in contact with the region of tissue;
delivering at least a first light illumination signal from the tissue interface region into the region of tissue in contact with the tissue interface region;
collecting at least a first captured light signal from the region of tissue at the tissue interface region in response to the first light illumination signal delivered into the region of tissue;
processing at least one measured parameter of at least the first captured light signal and producing a result that is useful in diagnosing the property of the region of tissue;
monitoring a pressure at the tissue interface region in contact with the region of tissue;
comparing the monitored pressure against a pressure threshold criteria; and
producing the result only based upon the monitored pressure meeting the pressure threshold criteria.

25. A medical diagnostic system, comprising:
an optical measurement system configured to measure at least one light scattering property or light absorption property of tissue;
a data analysis algorithm; and
a processor coupled to the data analysis algorithm;
wherein the data analysis algorithm run by the processor is configured to analyze data related to the measured optical property or properties in a manner that provides output information that is useful in diagnosing a property of the tissue; and
wherein the data analysis algorithm adjusts the analysis and output information based upon physical characteristics of the patient input by the user.
